SEQUENCE LISTING (52 sequences)

SEQ ID NO: 1 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 2 - protein, 9 aa, Severe acute respiratory syndrome coronavirus 2
YLQPRTFLL

SEQ ID NO: 3 - protein, 9 aa, Severe acute respiratory syndrome coronavirus 2
SPRWYFYYL

SEQ ID NO: 4 - protein, 15 aa, Severe acute respiratory syndrome coronavirus 2
LLLLDRLNQLESKMS

SEQ ID NO: 5 - protein, 9 aa, Severe acute respiratory syndrome coronavirus 2
LLLDRLNQL

SEQ ID NO: 6 - protein, 9 aa, Severe acute respiratory syndrome coronavirus 2
QYIKWPWYI

SEQ ID NO: 7 - protein, 9 aa, Severe acute respiratory syndrome coronavirus 2
KTFPPTEPK

SEQ ID NO: 8 - protein, 10 aa, Severe acute respiratory syndrome coronavirus 2
MEVTPSGTWL

SEQ ID NO: 9 - protein, 9 aa, Severe acute respiratory syndrome coronavirus 2
FTSDYYQLY

SEQ ID NO: 10 - protein, 9 aa, Severe acute respiratory syndrome coronavirus 2
SIIAYTMSL

SEQ ID NO: 11 - protein, 9 aa, Severe acute respiratory syndrome coronavirus 2
FIAGLIAIV

SEQ ID NO: 12 - protein, 9 aa, Severe acute respiratory syndrome coronavirus 2
LTDEMIAQY

SEQ ID NO: 13 - protein, 9 aa, Severe acute respiratory syndrome coronavirus 2
IPRRNVATL

SEQ ID NO: 14 - protein, 9 aa, Severe acute respiratory syndrome coronavirus 2
GMSRIGMEV

SEQ ID NO: 15 - protein, 9 aa, Severe acute respiratory syndrome coronavirus 2
NYNYLYRLF

SEQ ID NO: 16 - protein, 9 aa, Severe acute respiratory syndrome coronavirus 2
FLLNKEMYL

SEQ ID NO: 17 - protein, 9 aa, Severe acute respiratory syndrome coronavirus 2
SMWALIISV

SEQ ID NO: 18 - protein, 9 aa, Severe acute respiratory syndrome coronavirus 2
FPRGQGVPI

SEQ ID NO: 19 - protein, 9 aa, Severe acute respiratory syndrome coronavirus 2
ALWEIQQVV

SEQ ID NO: 20 - protein, 9 aa, Severe acute respiratory syndrome coronavirus 2
VYIGDPAQL

SEQ ID NO: 21 - protein, 9 aa, Severe acute respiratory syndrome coronavirus 2
YLDAYNMMI

SEQ ID NO: 22 - protein, 9 aa, Severe acute respiratory syndrome coronavirus 2
ALSKGVHFV

SEQ ID NO: 23 - protein, 10 aa, Severe acute respiratory syndrome coronavirus 2
KLPDDFTGCV

SEQ ID NO: 24 - protein, 9 aa, Severe acute respiratory syndrome coronavirus 2
RLNEVAKNL

SEQ ID NO: 25 - protein, 9 aa, Severe acute respiratory syndrome coronavirus 2
VLNDILSRL

SEQ ID NO: 26 - protein, 9 aa, Severe acute respiratory syndrome coronavirus 2
YYQLYSTQL

SEQ ID NO: 27 - protein, 15 aa, Severe acute respiratory syndrome coronavirus 2
FPRGQGVPINTNSSP

SEQ ID NO: 28 - protein, 14 aa, Severe acute respiratory syndrome coronavirus 2
QTVTKKSAAEASKK

SEQ ID NO: 29 - protein, 15 aa, Severe acute respiratory syndrome coronavirus 2
ILLNKHIDAYKTFPP

SEQ ID NO: 30 - protein, 15 aa, Severe acute respiratory syndrome coronavirus 2
NVTWFHAIHVSGTNG

SEQ ID NO: 31 - protein, 15 aa, Severe acute respiratory syndrome coronavirus 2
KVGGNYNYLYRLFRK

SEQ ID NO: 32 - protein, 15 aa, Severe acute respiratory syndrome coronavirus 2
FYSKWYIRVGARKSA

SEQ ID NO: 33 - protein, 8 aa, Severe acute respiratory syndrome coronavirus 2
GYLQPRTF

SEQ ID NO: 34 - protein, 9 aa, Severe acute respiratory syndrome coronavirus 2
KNKCVNFNF

SEQ ID NO: 35 - protein, 9 aa, Severe acute respiratory syndrome coronavirus 2
FPQSAPHGV

SEQ ID NO: 36 - protein, 15 aa, Severe acute respiratory syndrome coronavirus 2
VTWFHAIHVSGTNGT

SEQ ID NO: 37 - protein, 15 aa, Severe acute respiratory syndrome coronavirus 2
CYDYCIPYNSVTSSI

SEQ ID NO: 38 - protein, 15 aa, Severe acute respiratory syndrome coronavirus 2
EPIYDEPTTTTSVPL

SEQ ID NO: 39 - protein, 15 aa, Severe acute respiratory syndrome coronavirus 2
QFAFACPDGVKHVYQ

SEQ ID NO: 40 - protein, 8 aa, Severe acute respiratory syndrome coronavirus 2
GFSALEPL

SEQ ID NO: 41 - protein, 8 aa, Severe acute respiratory syndrome coronavirus 2
RTLSYYKL

SEQ ID NO: 42 - protein, 8 aa, Severe acute respiratory syndrome coronavirus 2
RGWIFGTT

SEQ ID NO: 43 - protein, 9 aa, Severe acute respiratory syndrome coronavirus 2
PIGINITRF

SEQ ID NO: 44 - protein, 8 aa, Severe acute respiratory syndrome coronavirus 2
AAYYVGYL

SEQ ID NO: 45 - protein, 8 aa, Severe acute respiratory syndrome coronavirus 2
EIYQAGST

SEQ ID NO: 46 - protein, 8 aa, Severe acute respiratory syndrome coronavirus 2
VVVLSFEL

SEQ ID NO: 47 - protein, 9 aa, Severe acute respiratory syndrome coronavirus 2
CVNFNFNGL

SEQ ID NO: 48 - protein, 9 aa, Severe acute respiratory syndrome coronavirus 2
DLLFNKVTL

SEQ ID NO: 49 - protein, 9 aa, Severe acute respiratory syndrome coronavirus 2
YLYALVYFL

SEQ ID NO: 50 - DNA, 1551 bp, synthetic construct
  source 1..1551: mol_type = other DNA
atggaggcggcggtcgctgctccgcgtccccggctgctcctcctcgtgctggcggcggcggcggcggcggcggcggcgctgctcccgggggcgacggcgttacagtgtttctgccacctctgtacaaaagacaattttacttgtgtgacagatgggctctgctttgtctctgtcacagagaccacagacaaagttatacacaacagcatgtgtatagctgaaattgacttaattcctcgagataggccgtttgtatgtgcaccctcttcaaaaactgggtctgtgactacaacatattgctgcaatcaggaccattgcaataaaatagaacttccaactactggccctttttcagtaaagtcatcacctggccttggtcctgtgacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaa

SEQ ID NO: 51 - protein, 15 aa, Severe acute respiratory syndrome coronavirus 2
QRNAPRITFGGPSDS

SEQ ID NO: 52 - protein, 15 aa, synthetic construct
GGGGSGGGGSGGGGS

(19) United States
(12) Patent Application Publication
(10) Pub. No.: US 2026/0345780 A1
(43) Pub. Date: Nov. 5, 2026

(54) IMMUNOTHERAPY COMPOSITIONS AND METHODS OF USE

(71) Applicant: PyrOjas Inc., New York, NY (US)

(72) Inventor: Archis Bagati, New York, NY (US)

(21) Appl. No.: 19/123,360

(22) PCT Filed: Oct. 24, 2023
(86) PCT No.: PCT/US2023/035802

§ 371 Date: Apr. 22, 2025

Related U.S. Application Data
(60) Provisional application No. 63/421,050, filed on Oct. 31, 2022.
(60) Provisional application No. 63/437,196, filed on Jan. 5, 2023.

(51) Int. Cl. A61K 40/42; A61K 39/00; A61K 39/215; A61K 40/11; A61K 40/31; A61K 40/50; A61P 35/00

(52) U.S. Cl. CPC A61K 40/4203; A61K 39/215; A61K 40/11; A61K 40/31; A61K 40/4211; A61K 40/50; A61P 35/00; A61K 2039/5256; A61K 2039/585; C12N 2740/15043; C12N 2770/20022; C12N 2770/20034

(57) ABSTRACT

The disclosure leverages the therapeutic potential of autologous and allogenic cells, particularly cancer cells and T cells, which have been genetically modified or contacted with specific recombinant proteins, peptides, or nucleic acids encoding these entities. These modified cells are primed to elicit various immune and inflammatory responses in the tumor microenvironment, thereby bolstering the body's natural ability to recognize and destroy tumor cells.

### CROSS-REFERENCE TO RELATED APPLICATIONS

This application claims benefit of priority under 35 U.S.C. § 119(e) to U.S. Provisional Application Nos. 63/421,050 filed Oct. 31, 2022, and 63/437,196 filed Jan. 5, 2023. The disclosures of the prior applications are considered part of and are herein incorporated by reference in the disclosure of this application in their entirety.

### INCORPORATION OF SEQUENCE LISTING

The material in the accompanying sequence listing is hereby incorporated by reference into this application. The accompanying sequence listing xml file, name PY R01100-3WO_SL, was created on Oct. 20, 2023, and is 48 kb in size.

### FIELD OF THE DISCLOSURE

The present disclosure relates in general to the field of immunotherapy and more specifically to compositions and methods of developing cellular and viral immunotherapy which confers protective immunity against human cancers.

### BACKGROUND OF THE DISCLOSURE

While cancer vaccines are effective at preventing disease, they have limited therapeutic potential due to four major reasons. First, central and peripheral tolerance of the immune system. The immune system detects “self” antigens expressed by cancer cells and becomes tolerant towards them instead of killing them. Second, heterogeneity of tumor cells. Tumor cells that lack the antigen or have defective antigen presentation evade detection and killing by antigen specific immune cells. Third, the immune-privileged TIMEs, tumor immune microenvironment. Immunosuppressive molecules and cells in the TIME limit the anti-tumor immune response. And four, most patients with advanced cancers are immunocompromised which makes treating established malignancies challenging.

The disclosure describes the composition of the genetic and the chemical modifications used to create autologous or allogenic cellular immunotherapies, or viral immunotherapies which stimulate the patient's own immune system to detect abroad repertoire of cancer specific antigens and kill heterogeneous cancer cells. Patient derived cells, either cancer or immune, are modified genetically and/or chemically ex-vivo to generate cellular immunotherapies that overcome central and peripheral immune tolerance, increase tumor immunogenicity, remodel the immunosuppressive tumor immune microenvironment, and re-direct an existing pool of immune cells to kill cancer cells.

Cancer vaccine platforms include cellular vaccines, viral or bacterial vaccines, and molecular vaccines utilizing DNA, RNA, or peptides.

Autologous APCs, antigen presenting cells, specifically dendritic cells are either loaded with peptide antigen or transfected with antigen genes to create DC vaccines. The first FDA approved cancer vaccine sipuleucel-T (Provenge) is used for the treatment of metastatic castration-resistant prostate cancer (mCRPC). This vaccine, generated by enriching DCs after leukapheresis and activating them ex-vivo with a chimeric protein GM-CSF fused to the antigen PAP, are safe but have limited efficacy. Several other DC vaccine-based phase I and II trials with autologous DCs pulsed with cancer antigen peptide or transduced with an adenovirus encoding the antigen are ongoing.

Whole cell vaccines. Autologous and allogeneic whole cell vaccines have been investigated in phase II and phase III trials but have yet to receive FDA approval. Autologous tumor vaccines were clinically evaluated in the 1970s by administration of patient-derived tumor cells with an adjuvant or virus to stimulate a polyclonal immune response to TAAs, tumor-specific antigens. M ore recently, GVAX whole tumor cell vaccine genetically modified to secrete the immune stimulatory cytokine, GM-CSF, granulocyte-macrophage colony-stimulating factor has been evaluated in autologous and allogeneic settings. GVAX enhances the recruitment and maturation of hematopoietic precursors into professional antigen presenting (APC) dendritic cells (DC). However, even though GVAX induces immune responses and tumor regression in murine tumor models, its clinical efficacy is limited in prostate, melanoma, lung, and pancreatic cancer. Overall, GM-CSF has been widely used in cancer vaccine trials with limited results.

Microorganisms to stimulate an immune response or deliver tumor antigens. Heat-inactivated bacteria was first used by Coley to stimulate significant anti-tumor immune responses in patients with cancer. A live attenuated strain of Mycobacterium bovis, Bacillus Calmette-Guerin, has been used for over 35 years to treat bladder carcinoma. While several reports have rationalized how BCG induces an anti-tumor immune response, the precise cellular and molecular mechanisms remain unclear. Listeria, Salmonella, Lactococcus, and Shigella are other species of bacteria have been used as effective vaccine vectors as well. In pre-clinical models, treatment with attenuated strains of Listeria monocytogenes results in the internalization of the bacteria by APCs, and delivery of DNA- or RNA-encoded tumor antigens to induce potent anti-tumor immunity. In clinical trials, attenuated Listeria-based cancer vaccines have a favorable safety profile but limited efficacy.

In most cancers, the tumors express self-proteins which tolerize the immune system. Tumor antigen specific vaccines generally show poor antigenicity due to immune tolerance and fail to activate a robust, clinically meaningful antitumor immune response. Not surprisingly, vaccines are effective when used in a prophylactic setting but have limited efficacy in when the disease is well-established (therapeutic setting). Thus, to be efficacious, a cancer vaccine needs to overcome tolerance by either stimulating low affinity or rare TAA-reactive T cells that remain or stimulate novel TAA-reactive T cells. Adjuvants, activators of antigen presentation, and serial vaccinations that promote the expansion of tumor-antigen-reactive T cells, particularly low-affinity T cells are needed. Importantly, presentation of the complete repertoire of tumor antigens to the immune system is needed to hedge against tolerogenic epitopes.

The heterogeneity of cancer cells confers them with a significant survival advantage. Sub-populations of tumor cells evade baseline or therapy-induced immune surveillance and underlie relapses and poor patient survival outcomes. Therefore, a strategy is needed to enhance the antigenicity of heterogenous tumor cells and stimulate an immune response against the entire available repertoire of tumor antigens.

N on-whole cell TAA-specific vaccines, such as peptide or whole protein vaccines, or antigen-specific DNA or mRNA vaccines require knowledge of the pattern of expression of targetable tumor antigens, and their immunogenicity in cancer patients. The efficacy of these TAA-specific approaches relies heavily on the expression pattern and representation of target antigen in the patient's tumor. Problematically, the expression pattern of the antigen is often heterogeneous and the immunogenicity of the antigen, variable. Further most cancer vaccines aim to stimulate an immune response against one or a limited set of tumor antigens. It is not sufficient to stimulate an immune response against a limited set of predicted neo-antigens for various reasons. The expression of the entire tumor antigen repertoire is essential for activating an immune response against a broad repertoire of cancer antigens to target heterogeneous cancers.

Immunosuppressive cells and molecules in the TIME suppress anti-tumor immunity. Principally, the cytokine TGF-β, Transforming Growth Factor beta ligands TGF-β1, -β2, -β3 are the cause of the significant immunosuppression in the TIME. TGF-β is ubiquitously expressed in human cancers and correlates with disease progression and poor patient prognosis. It causes anergy of immune cells in the TIME, suppresses the cytotoxic potential of CD8⁺ and CD4⁺ T cells, and converts naïve T cells into immunosuppressive regulatory T cells. TGF-β also potently blocks maturation of DCs by GM-CSF, the expression co-stimulatory molecules, and MHC class II, important for presentation of antigens to CD4⁺ T cells. TGF-β directly inhibits antigen presentation capacity of macrophages and dendritic cells and limits the cytotoxic potential of cytotoxic CD8⁺ T cells and CD4⁺ T cells. Therefore, overcoming the immunosuppression induced by TGF-β in the TIME is critical to enable an effective anti-tumor immune response to persist. Neutralizing anti-TGF-β antibodies, RNAi, expression of a mutated TGF-β1 precursors or dominant negatives have been utilized to neutralize TGF-β activity.

Tertiary Lymphoid Structures are highly differentiated compartments responsible for coordinating and guiding the differentiation and proliferation of lymphocytes. Bone marrow and thymus are primary lymphoid organs where naïve B and T lymphocytes mature from immature hematopoietic precursors. Secondary lymphoid organs (SLOs) including the spleen, lymph nodes, and mucosal-associated lymphoid tissue (MALT), coordinate trafficking of lymphocytes and maintain immune tolerance. Tertiary lymphoid organs (TLOs) or tertiary lymphoid structures (TLS) are highly organized aggregates of lymphocytes that accompany chronic inflammation, persistent infections, autoimmune transplant rejection, and cancer. TLS are characterized by (i) a distinct T-lymphocytes rich zone enclosing a central B-cell rich area, similar to a germinal center; (ii) follicular dendritic cells (FDCs) and activated stromal mesenchymal cells (fibroblasts) (iii) plasma blasts and (iv) high endothelial venules (H EVs); blood vessels that promote the migration of naïve lymphocytes into SLOs. TLS express lymphoid associated chemokines and lymphotoxins (LTs), contain germinal centers (GCs) that are sites for in situ B cell differentiation, somatic hypermutation, oligoclonal expansion, and antibody production.

Chemokines are small (7-12 kDa) chemotactic polypeptides that direct lymphocyte recruitment and organize the architecture of lymphoid organs. Chemokines CXCL12, CXCL13, CCL19, and CCL21 are constitutively expressed in lymphoid organs where they regulate lymphocyte migration, and segregation of B and T cells into T cell zones and germinal centers. Lymphoid chemokines and LTs work in concert with numerous cytokines to shape the cellular microenvironment during tertiary lymphoid neogenesis. These may include, Th17-cytokines (IL-17, IL-22, IL-23), IL-21, IL-36 agonists, and IL-1 family of cytokines IL-la, IL-13, IL-18, and IL-33.

Cancers differ from foreign bacterial, or viruses in their antigenicity. While vaccines against viruses often result in activation and proliferation of over 5% antigen specific CD8⁺ T cells relative to total circulating CD8 T cells, vaccines against cancer antigens result in less than 1% antigen specific CD8⁺ T cells. For example, yellow fever and smallpox vaccines stimulate expansion of activated antiviral CD8⁺ T cells to 12.5% and 40% of total peripheral CD8 T cells, respectively. In contrast, PROSTVAC-VF, a metastatic prostate cancer vaccine targeting PSA induced expansion of antigen-specific T cells to only approximately 0.03% of the total CD8⁺ T cell population and was halted in phase III due to a lack of efficacy. The absolute T cell numbers and threshold of quality needed for tumor control remain unclear and seems to be dependent on antigen type, T cell receptor (TCR) affinity, tumor type, tumor microenvironment. Collectively, these data suggest that the quantity and quality of antigen-specific T cells must surpass a critical threshold to result in clinical benefit; presence of peripheral antigen-specific T cells is insufficient to predict efficacy.

### SUMMARY OF THE DISCLOSURE

The present disclosure relates to the field of cancer immunotherapy, offering a range of compositions and methods for the treatment of cancer and benign tumors, including leiomyomas.

In one embodiment, the present disclosure provides a composition including an autologous cancer cell from a target subject, wherein the autologous cancer cell includes: (a) a recombinant protein or peptide or mRNA encoding the recombinant protein or peptide that induces an immune response; (b) a recombinant protein or peptide or mRNA encoding the recombinant protein or peptide that induces an inflammatory cell death response; (c) a recombinant protein or peptide or mRNA encoding the recombinant protein or peptide that induces the activation and persistence of antigen-presenting cells and cytotoxic T cells within the microenvironment of a tumor; or (d) a recombinant protein or peptide or mRNA encoding the recombinant protein or peptide that induces the formation of tumor-associated tertiary lymphoid structures. In one aspect, the recombinant protein or peptide that induces an immune response is selected from one of the following proteins: (a) SARS-Cov-2 Spike (S); (b) SARS-Cov-2 Envelope (E); (c) SARS-Cov-2 Membrane (M); (d) SARS-Cov-2 Nucleocapsid (N); (e) SARS-CoV-2 Spike S-2P and RBD antigen; (f) SARS-Cov-2 ORF3a; (g) SARS-Cov-2 ORF7a; (h) SARS-Cov-2 ORF8; (i) SARS-Cov-2 Replicase 1AB; (j) Enhanced Green Fluorescent Protein (EGFP); or (k) Influenza Hemagglutinin (HA).

In one aspect, the recombinant protein or peptide that induces an inflammatory cell death response is selected from one of the following proteins: (a) Gasdermin D (GSDM D); (b) Gasdermin E (GSDM E); (c) Absent In Melanoma-2 (AIM2); (d) Interleukin 33 (IL33); (e) Thioredoxin-Interacting Protein (TXNIP); (f) Interleukin 1 Receptor Associated Kinase 1 (IRAK1); or (g) NLR Family Pyrin Domain Containing 3 (NLRP3).

In one aspect the recombinant protein or peptide that induces the activation and persistence of antigen-presenting cells and cytotoxic T cells within the microenvironment of a tumor and a recombinant protein or peptide that induces that induce the formation of tumor-associated tertiary lymphoid structures is selected from one of the following proteins: (a) Chemokine Ligand 9 (CXCL9); (b) Chemokine Ligand 10 (CXCL10); (c) Chemokine Ligand 12 (CXCL12); (d) Chemokine Ligand 13 (CXCL13); (e) C-C Motif Chemokine Ligand 19 (CCL19); (f) C-C Motif Chemokine Ligand 21 (CCL21); (g) Interleukin 1 Beta (IL1); (h) Interleukin 4 (IL4); (i) Interleukin 4 Receptor (IL4R); (j) Interleukin 21 (IL21); (k) Interleukin 22 (IL22); (1) Interleukin 22 Receptor (IL22R); (m) Interleukin 23; (n) Interleukin 13 (IL13); (o) Lymphotoxin Beta Receptor (LT R); (p) Beta-2-Microglobulin (B2M); (q) Programmed Death-Ligand 1 (PD-L1); (r) Integrin Associated Protein (CD47); (s) Transforming Growth Factor Beta Receptor I (TGFβRI)-ectodomain, soluble; (t) Transforming Growth Factor Beta Receptor II (TGFβRII)-ectodomain, soluble; (u) Transforming Growth Factor Beta Receptor III (TGFβRIII)-ectodomain, soluble; (v) a protein consisting of the ectodomains of one or more of Transforming Growth Factor Beta Receptor I (TGFβRI), Transforming Growth Factor Beta Receptor II (TGFβRII), or Transforming Growth Factor Beta Receptor III (TGFβRIII) in any permutation; (w) Mothers Against Decapentaplegic Homolog 2 (SMAD2); or (x) Mothers Against Decapentaplegic Homolog 3 (SMAD3).

In one aspect, the autologous cancer cell is contacted with a mixture of cytokines and chemokines. In one aspect, at least one of the cytokines is Tumor Necrosis Factor Alpha (TNFα) or Interferon Gamma (IFNγ).

In one embodiment, the present disclosure provides a pharmaceutical composition formulated for delivery with a lipid nanoparticles, electroporation, or other delivery mechanisms, including the autologous cancer cell.

In another embodiment, the present disclosure provides a method for treating cancer and benign tumors (including leiomyomas) in a subject, including the composition described above.

In a further embodiment, present disclosure provides a composition including an allogenic cell including: (a) a recombinant protein or peptide or mRNA encoding the recombinant protein or peptide that induces an immune response; (b) a recombinant protein or peptide or mRNA encoding the recombinant protein or peptide that induces an inflammatory cell death response; (c) a recombinant protein or peptide or mRNA encoding the recombinant protein or peptide that induces the activation and persistence of antigen-presenting cells and cytotoxic T cells within the microenvironment of a tumor; or (d) d) a recombinant protein or peptide or mRNA encoding the recombinant protein or peptide that induces the formation of tumor-associated tertiary lymphoid structures.

In one aspect, the recombinant protein or peptide that induces an immune response is selected from one of the following proteins: (a) SARS-Cov-2 Spike (S); (b) SARS-Cov-2 Envelope (E); (c) SARS-Cov-2 Membrane (M); (d) SARS-Cov-2 Nucleocapsid (N); (e) SARS-CoV-2 Spike S-2P and RBD antigen; (f) SARS-Cov-2 ORF3a; (g) SARS-Cov-2 ORF7a; (h) SARS-Cov-2 ORF8; (i) SARS-Cov-2 Replicase 1AB; (j) Enhanced Green Fluorescent Protein (EGFP); or (k) Influenza Hemagglutinin (HA).

In one aspect, recombinant protein or peptide that induces an inflammatory cell death response is selected from one of the following proteins: (a) Gasdermin D (GSDM D); (b) Gasdermin E (GSDM E); (c) Absent In Melanoma-2 (AIM2); (d) Interleukin 33 (IL33); (e) Thioredoxin-Interacting Protein (TXNIP); (f) Interleukin 1 Receptor Associated Kinase 1 (IRAK1); or (g) NLR Family Pyrin Domain Containing 3 (NLRP3).

In one aspect, the recombinant protein or peptide that induces the activation and persistence of antigen-presenting cells and cytotoxic T cells within the microenvironment of a tumor and a recombinant protein or peptide that induces that induce the formation of tumor-associated tertiary lymphoid structures is selected from one of the following proteins: (a) Chemokine Ligand 9 (CXCL9); (b) Chemokine Ligand 10 (CXCL10); (c) Chemokine Ligand 12 (CXCL12); (d) Chemokine Ligand 13 (CXCL13); (e) C-C Motif Chemokine Ligand 19 (CCL19); (f) C-C Motif Chemokine Ligand 21 (CCL21); (g) Interleukin 1 Beta (IL1); (h) Interleukin 4 (IL4); (i) Interleukin 4 Receptor (IL4R); (j) Interleukin 21 (IL21); (k) Interleukin 22 (IL22); (1) Interleukin 22 Receptor (IL22R); (m) Interleukin 23; (n) Interleukin 13 (IL13); (o) Lymphotoxin Beta Receptor (LTβR); (p) Beta-2-Microglobulin (B2M); (q) Programmed Death-Ligand 1 (PD-L1); (r) Integrin Associated Protein (CD47); (s) Transforming Growth Factor Beta Receptor I (TGFβRI)-ectodomain, soluble; (t) Transforming Growth Factor Beta Receptor II (TGFβRII)-ectodomain, soluble; (u) Transforming Growth Factor Beta Receptor III (TGFβRIII)-ectodomain, soluble; (v) a fusion protein consisting of the ectodomains of one or more of: Transforming Growth Factor Beta Receptor I (TGFβRI), Transforming Growth Factor Beta Receptor II (TGFβRII), or Transforming Growth Factor Beta Receptor III (TGFβRIII) in any permutation; (w) Mothers Against Decapentaplegic Homolog 2 (SMAD2); or (x) Mothers Against Decapentaplegic Homolog 3 (SMAD3).

In one aspect, the allogenic cell is contacted with an autologous cancer cell from a subject, wherein the autologous cancer cell is first contacted with a mixture of cytokines and chemokines.

In one embodiment, the present disclosure provides a pharmaceutical composition formulated for delivery using lipid nanoparticles, electroporation, or other suitable delivery mechanisms, including the allogenic cells described above.

In another embodiment, the present disclosure provides a method for treating cancer and benign tumors (including leiomyomas) in a subject, including administering to the subject the pharmaceutical composition described above.

In a further embodiment, the present disclosure provides a composition including a recombinant virus including: (a) a recombinant protein or peptide or mRNA encoding the recombinant protein or peptide that induces an immune response; (b) a recombinant protein or peptide or mRNA encoding the recombinant protein or peptide that induces an inflammatory cell death response; (c) a recombinant protein or peptide or mRNA encoding the recombinant protein or peptide that induces the activation and persistence of antigen-presenting cells and cytotoxic T cells within the microenvironment of a tumor; or (d) a recombinant protein or peptide or mRNA encoding the recombinant protein or peptide that induces the formation of tumor-associated tertiary lymphoid structures.

In one aspect, the recombinant nucleic acid encoding the recombinant protein or peptide that induces an immune response is selected from a gene encoding one of the following proteins: (a) SARS-Cov-2 Spike (S); (b) SARS-Cov-2 Envelope (E); (c) SARS-Cov-2 Membrane (M); (d) SARS-Cov-2 Nucleocapsid (N); (e) SARS-CoV-2 Spike S-2P and RBD antigen (f) SARS-Cov-2 ORF3a; (g) SARS-Cov-2 ORF7a; (h) SARS-Cov-2 ORF8; (i) SARS-Cov-2 Replicase 1AB; (j) Enhanced Green Fluorescent Protein (EGFP); or (k) Influenza Hemagglutinin (HA).

In another aspect, the recombinant nucleic acid encoding the recombinant protein or peptide that induces an inflammatory cell death response is selected from a gene encoding one of the following proteins: (a) Gasdermin D (GSDM D); (b) Gasdermin E (GSDM E); (c) Absent In Melanoma-2 (AIM2); (d) Interleukin 33 (IL33); (e) Thioredoxin-Interacting Protein (TXNIP); (f) Interleukin 1 Receptor Associated Kinase 1 (IRAK1); or (g) NLR Family Pyrin Domain Containing 3 (NLRP3).

In a further aspect, the recombinant protein or peptide that induces the activation and persistence of antigen-presenting cells and cytotoxic T cells within the microenvironment of a tumor and induce the formation of tumor-associated tertiary lymphoid structures is selected from one of the following proteins: (a) Chemokine Ligand 9 (CXCL9); (b) Chemokine Ligand 10 (CXCL10); (c) Chemokine Ligand 12 (CXCL12); (d) Chemokine Ligand 13 (CXCL13); (e) C-C Motif Chemokine Ligand 19 (CCL19); (f) C-C Motif Chemokine Ligand 21 (CCL21); (g) Interleukin 1 Beta (IL1); (h) Interleukin 4 (IL4); (i) Interleukin 4 Receptor (IL4R); (j) Interleukin 21 (IL21); (k) Interleukin 22 (IL22); (l) Interleukin 22 Receptor (IL22R); (m) Interleukin 23; (n) Interleukin 13 (IL13); (o) Lymphotoxin Beta Receptor (LT R); (p) Beta-2-Microglobulin (B2M); (q) Programmed Death-Ligand 1 (PD-L1); (r) Integrin Associated Protein (CD47); (s) Transforming Growth Factor Beta Receptor I (TGFβRI)-ectodomain, soluble; (t) Transforming Growth Factor Beta Receptor II (TGFβRII)-ectodomain, soluble; (u) Transforming Growth Factor Beta Receptor III (TGFβRIII)-ectodomain, soluble; (v) A ny fusion protein consisting of the ectodomains of one or more of the following: Transforming Growth Factor Beta Receptor I (TGFβRI), Transforming Growth Factor Beta Receptor II (TGFβRII), and Transforming Growth Factor Beta Receptor III (TGFβRIII) in any permutation; (w) Mothers Against Decapentaplegic Homolog 2 (SMAD2); or (x) Mothers Against Decapentaplegic Homolog 3 (SMAD3).

In one aspect, the present disclosure provides a pharmaceutical composition for delivery to a subject using lipid nanoparticles, electroporation, or other suitable delivery mechanisms, including the recombinant virus or delivery system described above.

In another aspect, the present disclosure provides a method for treating cancer and benign tumors (including leiomyomas) in a subject, including administering to the subject the pharmaceutical composition described above.

In a further aspect, the present disclosure provides a composition including an allogenic T cell, wherein the allogenic T cell includes: (a) a chimeric antigen receptor (CA R) or an antigen specific T cell receptor (TCR) that makes theT cells antigen specific; and (b) a recombinant protein or peptide or mRNA encoding the recombinant protein or peptide that induces the activation and persistence of antigen-presenting cells and cytotoxic T cells within the microenvironment of a tumor. (c) a recombinant protein or peptide or mRNA encoding the recombinant protein or peptide that induces that induce the formation of tumor-associated tertiary lymphoid structures.

In a still further aspect, the chimeric antigen receptor (CAR) or an antigen specific T cell receptor (TCR) is specific for one of the following proteins: (a) SARS-Cov-2 Spike (S); (b) SARS-Cov-2 Envelope (E); (c) SARS-Cov-2 Membrane (M); (d) SARS-Cov-2 Nucleocapsid (N); (e) SARS-CoV-2 Spike S-2P and RB D antigen; (f) SARS-Cov-2 OR F3a; (g) SARS-Cov-2 ORF7a; (h) SARS-Cov-2 ORF8; (i) SARS-Cov-2 Replicase 1AB; (j) Enhanced Green Fluorescent Protein (EGFP); or (k) Influenza Hemagglutinin (HA).

In one aspect, the recombinant protein or peptide that induces the activation and persistence of antigen-presenting cells and cytotoxic T cells within the microenvironment of a tumor and induce the formation of tumor-associated tertiary lymphoid structures is selected from one of the following proteins: (a) Chemokine Ligand 9 (CXCL9); (b) Chemokine Ligand 10 (CXCL10); (c) Chemokine Ligand 12 (CXCL12); (d) Chemokine Ligand 13 (CXCL13); (e) C-C Motif Chemokine Ligand 19 (CCL19); (f) C-C Motif Chemokine Ligand 21 (CCL21); (g) Interleukin 1 Beta (IL1); (h) Interleukin 4 (IL4); (i) Interleukin 4 Receptor (IL4R); (j) Interleukin 21 (IL21); (k) Interleukin 22 (IL22); (1) Interleukin 22 Receptor (IL22R); (m) Interleukin 23; (n) Interleukin 13 (IL13); (o) Lymphotoxin Beta Receptor (LT R); (p) Beta-2-Microglobulin (B2M); (q) Programmed Death-Ligand 1 (PD-L1); (r) Integrin Associated Protein (CD47); (s) Transforming Growth Factor Beta Receptor I (TGFβRI)-ectodomain, soluble; (t) Transforming Growth Factor Beta Receptor II (TGFβRII)-ectodomain, soluble; (u) Transforming Growth Factor Beta Receptor III (TGFβRIII)-ectodomain, soluble; (v) Any fusion protein including the ectodomains of one or more of: Transforming Growth Factor Beta Receptor I (TGFβRI), Transforming Growth Factor Beta Receptor II (TGFβRII), or Transforming Growth Factor Beta Receptor III (TGFβRIII) in any permutation; (w) Mothers Against Decapentaplegic Homolog 2 (SMAD2); or (x) Mothers Against Decapentaplegic Homolog 3 (SMAD3).

In one aspect, the present disclosure provides a pharmaceutical composition suitable for delivery to a subject using lipid nanoparticles, electroporation, or other suitable delivery mechanisms, including the allogenic cell described above.

In another aspect, the present disclosure provides a method for treating cancer and benign tumors (including leiomyomas) in a subject, including administering to the subject the pharmaceutical composition.

In a further aspect, the present disclosure provides a composition including a recombinant DNA, an mRNA, lipid nanoparticles, or an electroporation system, wherein the recombinant DNA or mRNA encodes a recombinant protein or peptide that induces an immune response.

In one aspect, the recombinant nucleic acid encoding the recombinant protein or peptide that induces an immune response is selected from the gene encoding one of the following proteins: (a) SARS-Cov-2 Spike(S); (b) SARS-Cov-2 Envelope (E); (c) SARS-Cov-2 Membrane (M); (d) SARS-Cov-2 Nucleocapsid (N); (e) SARS-CoV-2 Spike S-2P and RBD antigen (f) SARS-Cov-2 ORF3a; (g) SARS-Cov-2 ORF7a; (h) SARS-Cov-2 ORF8; (i) SARS-Cov-2 Replicase 1AB; (j) Enhanced Green Fluorescent Protein (EGFP); or (k) Influenza Hemagglutinin (HA).

In another aspect, the present disclosure provides a pharmaceutical composition for delivery to the target subject using lipid nanoparticles, electroporation, or other delivery mechanisms, including the recombinant DNA, mRNA, or delivery system described above.

In a further aspect, the present disclosure provides a method for treating cancer and benign tumors (including leiomyomas) in a subject, including administering to the subject the pharmaceutical composition described above.

### 639. BRIEF DESCRIPTION OF THE DRAWINGS

FIG. 1A is a schematic diagram illustrating the process and design to generate PyroCells™ cellular immunotherapies.

FIG. 1B is a schematic diagram illustrating the process and design to generate Allo-PyroCell™ immunotherapies.

FIG. 1C is a schematic diagram illustrating the process and design to generate PyroVir™ viral immunotherapies.

FIG. 2 is a schematic diagram illustrating the concept that challenges associated with therapeutic cancer vaccines and how PyroVax™ vector encoding PyroHIM™, PyroACT™, and PyroTIME™ can be used to overcome several of these challenges (right).

FIG. 3 is a schematic diagram illustrating PyroVax™ components PyroHIM™, PyroACT™, and PyroTIME™ in a non-viral expression plasmid, including PyroVax™ in a lentiviral vector.

FIG. 4 is a schematic diagram illustrating PyroVax™ components PyroHIM™, PyroACT™, and PyroTIME™ in a non-viral expression plasmid, including PyroVax™ in an AAV vector.

FIG. 5 is a schematic diagram illustrating PyroVax™ components PyroHIM™, PyroACT™, and PyroTIME™ in a non-viral expression plasmid, including PyroVax™ in an AAV vector.

FIG. 6 is a schematic diagram illustrating PyroVax™ components PyroHIM™ PyroACT™, and PyroTIME™ in a non-viral expression plasmid, including a backbone.

FIG. 7A is a graph illustrating contour plots showing expression of indicated proteins in human TNBC cells transduced with lentiviral vectors encoding the indicated genes.

FIG. 7B is a graph illustrating quantification of data from FIG. 7A.

FIG. 7C is a set of graphs illustrating contour plots showing expression levels of indicated constructs using a non-viral approach.

FIG. 7D is a set of graphs illustrating flow plots showing the tetramer positive CD8⁺ T cells present in the blood of the donor described on the left.

FIG. 7E is a chart describing a certificate of analysis highlighting critical information about the donor from who the T cells shown in FIG. 7D and FIG. 7E were isolated.

FIG. 7F is a set of graphs illustrating A IFNγ assay (top) and a cytotoxicity assay (bottom) testing the cellular response to peptides encoding the T cells specific-reactive peptide.

FIG. 8A is a set of bioluminescent images illustrating the immunological rejection of PyroCells™ over time. By three weeks there is no detectable presence of PyroCells™ (left). On Day 22, vaccinated mice were challenged with parental tumor (red) or not (blue) and compared to unvaccinated controls which also received the same parental tumor. While vaccinated mice were protected from tumor challenge, all unvaccinated mice succumb to disease and died. Day 28 is 7 days after tumor challenge.

FIG. 8B is a line graph quantifying the bioluminescence signal in FIG. 8A. Data is represented as mean and standard deviation.

FIG. 9A and FIG. 9B are schematic representations of the PyroTIMER construct, highlighting the fusion of ectodomains of TGFβRI and TGFβRII, integrated into JurkatT cells.

FIG. 9C is a set of graphs illustrating a co-culture killing assay using PyroTIMER CD19CAR-T cells and CD19 antigen-expressing Raji lymphoma cells. Displayed is the increased cytotoxicity of PyroTIMER CD19 CAR-T cells compared to control groups in the presence of 100 pM TGF-β1.

FIG. 9D is a set of graphs illustrating stability assays showing the persistence of PyroTIMER and CD19 CAR expression in Jurkat T cells over time, post-transduction.

FIG. 9E is a graphical representation of the effector-to-target ratios used in the co-culture killing assay, highlighting the superior killing efficacy of PyroTIMER CD19 CAR-T cells across all ratios.

FIG. 10A is a set of graphs illustrating generation and functional characterization of PyroTIMER CD19 CAR-T cells using primary human CD3⁺ T cells, including contour flow plots showing purity of enrichment of CD3⁺ T cells from human donor PBMCs.

FIG. 10B is a graph illustrating quantification of FIG. 10A.

FIG. 10C is a set of graphs illustrating contour plots showing the frequency of CD4⁺ (top) and CD8⁺ (bottom) PyroTIMER CD19 CAR-T cells following enrichment by cell sorting.

FIG. 10D is a set of graphs illustrating contour flow plots showing the frequency of CD4⁺ and CD8⁺ among the CD3⁺ T cells.

FIG. 10E is a graph illustrating quantification of FIG. 10D.

FIG. 10F is a set of graphs illustrating contour flow plots showing the generation of CD19 CAR-T cells at high efficiency after chemical selection (puromycin) and cell sorting (RFP).

FIG. 10G is a graph illustrating the number of live CD19 expressing Raji lymphoma cells, 24 hrs after being in co-culture with either CD19 CAR-T cells (CAR) or PyroTIMER CD19 CAR-T cells (PyroTIMER CAR) at indicated effector to target (E:T) ratios in the presence of TGF β1.

FIG. 10H is a set of graphs illustrating the percentage of CD69⁺ activated T cells in FIG. 10G. Un-transduced T cells were used as controls. Experiments was replicated twice in technical triplicates. Data is a percentage of total and represented as an average+/−S.E.M. An unpaired two-tailed student t test was used to compared the killing (in G) and the CD69⁺ cells (in H) in either co-culture (CAR vs PyroTIMER CAR) at specific E:T ratios. A p<0.05 was considered statistically significant.

FIG. 11A and FIG. 11B area set of images and a graph illustrating results of an in vivo study using NOD.Cg-PrkdcscidIL2Rgtm1Wjl/Sz mice illustrating the efficacy of PyroTIMER CAR-T cells against CD19⁺ Raji lymphoma disease burden. The comparative tumor growth curves and survival rates between PyroTIMER CAR-T cell-treated mice and traditional CD19 CAR-T cell-treated mice are showcased.

### DETAILED DESCRIPTION OF THE DISCLOSURE

The present disclosure relates to the field of cancer immunotherapy, offering a range of compositions and methods for the treatment of cancer and benign tumors, including leiomyomas. The disclosure leverages the therapeutic potential of autologous and allogenic cells, particularly cancer cells and T cells, which have been genetically modified or contacted with specific recombinant proteins, or nucleic acids encoding the recombinant proteins or peptides. These modified cells are primed to elicit various immune and inflammatory responses in the tumor microenvironment, thereby bolstering the body's natural ability to recognize and destroy tumor cells.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods of the type described herein which will become apparent to those persons skilled in the art upon reading this disclosure and so forth.

As used herein, the term “and/or” includes any and all combinations of one or more of the associated listed items.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

The disclosure encompasses autologous cancer cells modified to express or be contacted with entities that induce immune responses, inflammatory cell death, the activation and persistence of antigen-presenting cells and cytotoxic T cells in the tumor milieu, and the formation of tumor-associated tertiary lymphoid structures. Such autologous cells include cells where the immune response is triggered by proteins produced by viruses like SARS-CoV-2 or other entities like EGFP and Influenza Hemagglutinin. Autologous cells of the present disclosure also include primed to induce inflammatory cell death by targeting or inhibiting proteins such as Gasdermins, AIM2, and IL33. Further autologous cells include cells enhanced to bolster the activation of immune cells and the formation of lymphoid structures in the tumor environment, leveraging proteins or inhibitors like CXCL9, CCL21, IL1P, and PD-L1, among others.

A Ilogenic cells, including T cells, are modified similarly to the autologous cells, allowing for a broader applicability in treatments. Recombinant viruses are engineered to deliver the same therapeutic entities directly into the tumor. Recombinant DNAs, mRNAs, lipid nanoparticles, or electroporation systems encoding specific proteins or peptides, especially those inducing an immune response, are described in the present disclosure. Additionally, the disclosure provides pharmaceutical compositions suitable for delivery to the subject using mechanisms like lipid nanoparticles and electroporation, ensuring efficient uptake and functionality of the therapeutic entities. Lastly, the disclosure offers methods for treating malignancies and benign tumors in subjects using the described compositions. Overall, this disclosure provides for innovative, targeted, and personalized therapeutic approaches in the fight against cancer, leveraging the body's immune system's power and specificity.

Immune cells continuously survey the body for pathogens like bacteria and viruses, or normal cells that might be becoming cancerous and kills them. However, in subjects who are immunocompromised, the heterogeneity of tumor cells allows for sub-populations of cells to evade detection and killing by immune cells and establish disease. These immune evasive tumor cells either do not present antigens that are vulnerable to detection by immune cells or completely halt antigen presentation. Since these cancer cells express most of the proteins and antigens that normal cells express, the immune system recognizes the cell as “self” and becomes “tolerant” of the cancer cells. These cancer cells disguise as “self”, avoid expressing vulnerable antigens, and secrete immunosuppressive molecules into the microenvironment, all of which compromise the ability of immune cells to infiltrate the tumor and kill the cancer cell. This immunosuppressive tumor immune microenvironment recruits immunosuppressive cells which turns the TIME into an immune privileged site allowing the tumor to grow unchecked.

This disclosure describes a novel autologous and allogeneic cellular immunotherapy, and a viral immunotherapy which break central and peripheral immune tolerance against tumor antigens, enhances the immunogenicity of cancer cells, and remodels the immunosuppressive TIME. The approach emphasizes the presentation of the entire available repertoire of subject tumor specific antigens to activate an anti-tumor immune response against a broad repertoire of cancer specific antigens. Notably, this approach redirects an existing repository of anti-viral immune cells to treat cancer. This approach is important because subjects with cancer are often immunocompromised and do not contain a robust repository of naïve immune cells to develop a significant anti-tumor immune response.

The term “isolated” indicates that the cell or cells, or the peptide(s) or nucleic acid molecule(s) has/have been removed from its/their normal physiological environment, e.g. a natural source, or that a peptide or nucleic acid is synthesized. Use of the term “isolated” indicates that a naturally occurring sequence has been removed from its normal cellular (i.e., chromosomal) environment. Thus, the sequence may be in a cell-free solution or placed in a different cellular environment. An isolated cell or isolated cells may for instance be included in a different medium such as an aqueous solution than provided originally, or placed in a different physiological environment. Typically, isolated cells, peptides or nucleic acid molecule(s) constitute a higher fraction of the total cells, peptides or nucleic acid molecule(s) present in their environment, e.g., solution/suspension as applicable, than in the environment from which they were taken.

The terms “treatment” and “treating” as used herein, refer to a prophylactic or preventative measure having a therapeutic effect and slowing down, or at least partially alleviating or abrogating an undesirable condition in the organism of a subject. Those in need of treatment include those already with the condition as well as those prone to having the condition or those in whom the condition is to be prevented (prophylaxis). Generally, a treatment reduces, stabilizes, or inhibits progression of a symptom that is associated with the presence and/or progression of an undesirable condition. The term “therapeutic effect” refers to the inhibition or activation of factors causing or contributing to the undesirable condition. A therapeutic effect relieves to some extent one or more of the symptoms of an undesirable condition or disease. The term “undesirable condition” refers to a function in the cells or tissues of an organism that deviates from the optimal functions in that organism.

As used herein, the term “administer” refers to any way a treatment is given to a patient or subject. “Administer” refers to any mode of transferring, delivering, introducing, or transporting matter such as a compound, e.g. a pharmaceutical compound, or other agent such as an antisense oligonucleotide, to a subject. Administering can be accomplished via topical, intravenous, intramuscular, systemic, oral, or parenteral methods.

The present disclosure provides the composition of and methods to develop an autologous cellular immunotherapy, namely PyroCells™. These are composed of patient derived (autologous) tumor cells which are modified ex-vivo to express the PyroVax™ vector and chemically modified with PyroStim™. When re-administered back into the same patient, PyroCells™ activate an immune response against a broad repertoire of tumor specific antigens conferring longterm recurrence free survival in patients with advanced solid and hematological cancers.

Another embodiment of the present disclosure provides the composition of and methods to develop auto-allogeneic cellular immunotherapies namely, A Ilo-PyroCell™, which are created by combining ex-vivo PyroStim™ treated autologous patient tumor derived cells with allogeneic cells (normal or transformed) which have been modified ex-vivo to express the PyroVax™ vector. Allo-PyroCell™ activate a polyclonal immune response against a broad repertoire of tumor specific antigens conferring long-term recurrence free survival. Notably, this allogeneic approach overcomes several manufacturing, technical, and cost challenges associated with an autologous approach.

Another embodiment of the present disclosure provides the composition of and methods to develop viral immunotherapies, namely, PyroVir™. The virus is delivered intratumorally and incorporate the components of PyroVax™ namely, PyroHIM™, PyroAct™, PyroTIME™, as well as PyroStim™ into the tumor to activate a polyclonal immune response against a broad repertoire of tumor specific antigens conferring long-term recurrence free survival.

The following section summarizes the three major components of the PyroVax™ vector namely, PyroHIM™, PyroAct™, and PyroTIME™, and the composition of the chemicals with relevant doses which make up PyroStim™. It also summarizes the methods to use these components to create PyroCells™, A Ilo-PyroCells™, and PyroVir™.

The present disclosure provides the composition of nucleotide sequences used to generate PyroVax™ vector. The PyroVax™ vector is comprised of nucleic acids encoding three essential ORFs, open reading frames, among various other regulatory components. These include: i) PyroHIM™ a highly immunogenic molecule expressed at supraphysiological levels which are non-self and have high antigenic potential, ii) PyroAct™, an activator of inflammatory immunogenic cell death, and iii) PyroTIME™, a remodeler of the immunosuppressive TIME, tumor immune microenvironment. These components of PyroVax™ can be operably linked together or not in various permutations, on the same or different vector, and under the control of the same or different promoter.

TABLE 1
showing PyroVax ™ components:

| PyroHIM ™ | PyroACT ™ | PyroTIME ™ |
|---|---|---|
| Epitopes from SARS-Cov-2_Spike glycoprotein (S) | GSDMD | CXCL13 |
| Epitopes from SARS-Cov-2_Envelope protein (E) | GSDMD-NT | CXCL12 |
| Epitopes from SARS-Cov-2_Membrane protein (M) | GSDME | CCL19 |
| Epitopes from SARS-Cov-2_Nucleocapsid | AIM2 | CCL21 |
| phosphoprotein (N) | | |
| Epitopes from SARS-CoV-2 Spike S-2P antigen | IL33 | IL1β |
| Epitopes from SARS-CoV-2 RBD | TXNIP | CXCL9 |
| Epitopes from Enhanced Green Fluorescent Protein | IRAK1 | CXCL10 |
| Epitopes from Influenza HA antigens | NLRP3 | B2M |
| | | CD274 |
| | | CD47 |
| | | STGFβ |
| | | RI/II/III |
| | | SMAD2/3 |
| | | IL13 |
| | | IL4R |
| | | IL4 |
| | | IL22 |
| | | IL22R |
| | | LTβR |

The present disclosure describes a method of making an autologous cellular immunotherapy (PyroCell™) including:
a) Harvesting the surgically resected tumor tissue in the sterile container and ship to PyroLabs™
b) Dissociation of the tumor mechanically and chemically into a single cell suspension.
c) Enrichment of tumor cells using antibody mediated negative selection.
d) Electroporation or viral transduction of autologous tumor cells to incorporate PyroVax™ vector.
e) Chemical selection to enrich for PyroVax™ expressing PyroCells™.
f) Enhancement of antigen presentation with PyroStim™
g) Treat the subject or freezing the immunotherapy at −80 C for 5 years.

The present disclosure describes a method of making a combination of an autologous and allogeneic cellular immunotherapy (Allo-PyroCell™) including:
a) Harvesting the surgically resected autologous tumor tissue in the sterile container.
b) Dissociation of the tumor mechanically and chemically into a single cell suspension.
c) Enrichment of tumor cells using antibody mediated negative selection.
d) Stimulation of antigen dose, and presentation by autologous tumor cells using PyroStim™, a combination dose of IFNγ and TNFα to stimulate MHC-I and MHC-II dependent antigen presentation.
h) Mix autologous tumor cells from above (4) with allogeneic cells which have been previously electroporated or virally transduction to incorporate PyroVax™ vector.
i) Freezing the immunotherapy at −80 C.

The present disclosure describes a method of making a cancer-specific viral immunotherapy (PyroVir™) including:
a) Generation of AAV and/or onco-specific viruses (HSV) containing PyroVax™ vectors for intra-tumoral delivery.
b) Chemical selection of genetically modified virus expressing PyroVax™, capable of infecting and propagating in cancer cells in vivo.
c) Freezing the immunotherapy at −80 C.

### Central and Peripheral Immune Tolerance Limits Anti-Tumor Immunity

The present disclosure describes a method of redirecting an immune response generated in a prophylactic setting towards cancer cells in a therapeutic setting. Specifically, the disclosure describes the composition of the genetic and chemical modifications and the methods used to generate autologous or allogeneic cellular immunotherapies which re-direct the immune cells that detect SARS-Cov-2 antigens and control viral infection to detect cancer antigens and kill cancer cells.

This approach takes advantage of the fact that a large majority of people have been and will be exposed to, infected by, recover from, or vaccinated against SARS-Cov-2. The available pool of anti-viral immune cells including CD8⁺ T cells, CD4⁺ T cells, and B cells, can be therefore re-directed to detect cancer antigens and kill cancer cells.

### Heterogeneity Allows Immune Evasion by Tumor Cells

The present disclosure describes a method to present the entire available subject specific neoantigen matrix by using subject-derived whole tumor cells. Autologous or allogeneic cells are modified ex-vivo such that when introduced into the subject, these cellular immunotherapies stimulate an immune response or redirect an existing immune repository to detect cancer specific antigens and kill cancer cells.

In other embodiments of the present disclosure, cells genetically modified to manufacture the cellular immunotherapy may be autologous, expansion of cells derived from a xenograft of the autologous tumor cells, allogeneic tumor cells, allogeneic tumor cells expanded in xenografts, or a combination of one or more.

In other embodiments of the present disclosure, allogenic cells are established from normal or transformed cell lines, or normal fibroblasts, or endothelial cells, or immortalized cell lines.

### Lack of Immunogenicity of Tumors

The present disclosure describes a method to significantly increase tumor immunogenicity by introducing a HIM, highly immunogenic molecules using PyroHIM™.

In one aspect, HIMs include full length proteins or immunogenic regions selected from the proteins including but not limited to enhanced green fluorescent protein (eGFP), the full-length protein or peptide derivatives of the SARS-CoV-2 (accession number NC_045512.2) structural proteins, including the spike glycoprotein (S), envelope protein (E), membrane protein (M), and nucleocapsid phosphoprotein (N), among others (see table 1).

Patients with cancer are immunocompromised due to various reasons (1) and do not have an available robust repository of immune cells to control aggressive solid human cancers. However, patients with cancer who have recovered from a SARS-Cov-2 infection or have been vaccinated with any of the approved vaccines against SARS-Cov-2, contain a robust pool of SARS-Cov-2 antigen specific immune cells. The disclosure described here re-directs or repurposes the immune response against SARS-CoV-2 towards killing cancer cells.

In one embodiment of PyroHIM™, the full-length protein or immunogenic peptides produced by the SARS-Cov-2 virus are encoded as PyroHIMs™ in the PyroVax™ construct operably linked with an activator of pyroptotic cell death (PyroAct™), and a remodeler of the TIME, tumor immune microenvironment (PyroTIME™).

In various aspects, PyroHIMs™ encode for the full-length protein(s) or peptide(s) of the SARS-CoV-2 (accession number NC_045512.2) structural proteins, including the spike glycoprotein (S), envelope protein (E), membrane protein (M), and nucleocapsid phosphoprotein (N). Notably, the immunogenic peptides are used alone, or in combination with other peptide fragments connected with linkers or not to enhance immunogenicity.

In one aspect of the disclosure, the epitopes used to develop the FDA approved vaccinations by Moderna and Pfizer among other vaccine manufacturers are used. These include the SARS-CoV-2 Spike S-2P antigen and the SARS-CoV-2 RBD antigen.

In one aspect of the disclosure, the activation of pyroptosis is achieved using SARS-Cov-2 antigens ORF3a and envelope protein (E) which are direct inducers of pyroptosis.

PyroCells™ containing PyroVax™ vectors encoding SARS-Cov-2 specific epitopes as PyroHIMs™, redirects the immune response generated against SARS-Cov-2 to treat cancer. PyroCells™ cellular immunotherapies are effective in both prophylactic and therapeutic settings, able to control disease, and significantly extend overall survival.

PyroCells™ containing PyroVax™ vectors encoding SARS-Cov-2 specific epitopes as PyroHIMs™, activate a pre-existing pool of anti-viral cytotoxic CD8⁺ and CD4⁺ T cells and redirect it against cancer. In one aspect, PyroHIM™ epitopes are linked to a reporter molecule to enable supraphysiological expression, detection, and enrichment of autologous or allogeneic PyroCells™ that express the PyroVax™ vector.

PyroCells™ containing PyroVax™ vectors encoding SARS-Cov-2 specific epitopes as PyroHIMs™, are operably linked together or not, derived from reactive T and B cell epitopes most prevalent in humans exposed to or vaccinated against SARS-Cov-2.

### EGFP, Enhanced Green Fluorescent Protein

AAB 02572: 239 aa, Accession: AAB 02572, Version: AAB 02572.1 (SEQ ID NO:1)
1 mvskgeelftgvvpilveld gdvnghkfsv sgegegdaty gkltlkficttgklpvpwpt
61 lvttltygvq cfsrypdhmk qhdffksamp egyvqertif fkddgnyktr aevkfegdtl
121 vnrielkgid fkedgnilgh kleynynshn vyimadkqkn gikvnfkirh niedgsvqla
181 dhyqqntpig dgpvllpdnh ylstqsalsk dpnekrdhmv llefvtaagi tlgmdelyk

In one embodiment of PyroHIM™, the full-length protein or immunogenic peptides such as EGFP or a portion thereof are encoded as antigens in the PyroVax™ construct operably linked with an activator of inflammatory cell death (PyroAct™), and a remodeler of the TIME, tumor immune microenvironment (PyroTIME™).

PyroCells™ cellular immunotherapies containing EGFP as a PyroHIM™ at supraphysiological concentrations, stimulates an immune response against the immunodominant antigen GFP, and notably against several less dominant cancer cell specific antigens. This results in the activation of a broad repertoire of cancer-antigen specific T cells which recognize a diverse set of tumor antigens and control established heterogeneous disease. GFP is a model non-self-antigen to test the utility of PyroHIMs™ in PyroVax™ because well-established in vitro and in vivo tools and model systems utilizing GFP as an antigen exist and are well validated (4, 5).

### Immunosuppressive Molecules and Cells in the TIME, Tumor Immune Microenvironment

PyroTIME™ is the component of the PyroVax™ vector that encodes for the molecules which remodel the immunosuppressive TIMEs by promoting tumor antigen presentation by APCs and killing by cytotoxic immune cells. PyroTIME™ works by suppressing immunosuppressive molecules and cells in the TIME and promotes an environment that generates TS-TLS, tumor specific-tertiary lymphoid structures. Specifically, PyroTIME™ inactivates the expression of specific genes and/or increase the expression of specific cytokines, chemokines, and soluble receptor antagonists which promote the recruitment of T cells (organized into T cell zones), B-cells (organized as a germinal center), and follicular dendritic cells giving rise to tumor specific tertiary lymphoid structures.

The present disclosure describes the composition of and methods to remodel the immunosuppressive TIME by suppressing the levels of the immunosuppressive cytokine, TGF-β in the TIME. PyroTIME™ encodes for competitive antagonists of TGFβ including soluble TGF-β receptors (TGFβRII or TGFβRIII), or dominant negative receptors.

In another aspect, PyroTIME™ encodes guide RNAs which inactivate specific TGFρ signaling genes including SMAD2/3, TGFβRII, TGFβRIII, among others (see table 1, PyroTIME™). In one embodiment of the present disclosure, the gRNA (crRNA+trRNA tracer RNA) and Cas9 are complexed as RNPs and electroporated or nucleofected into host cells. In another embodiment of the present disclosure, the gRNA and Cas9 are encoded in an expression vector.

In another aspect, PyroTIME™ is composed of nucleotide sequences which encode for genes that express soluble or dominant negative TGFβRII and/or TGFβRIII to inhibit the levels of TGFβ in the TIME.

Another embodiment of the present disclosure describes the composition of and methods to remodel the immunosuppressive TIME to promote increased intra-tumoral infiltration and persistence of immune cells using PyroTIME™. Specifically, the disclosure describes the composition of PyroTIME™, DNA sequences encoding for molecules CX CL 13 and CCL19 which promote recruitment of B cells, dendritic cells, cytotoxic T cells, formation of germinal centers, T cell zones, and finally tertiary lymphoid structures containing follicular dendritic cells.

The present disclosure describes PyroTIME™, a component of PyroVax™ which remodels the immunosuppressive TIME and promotes the formation of tumor specific TLSs, Tertiary Lymphoid Structures for durable anti-tumor immune surveillance.

PyroTIME™ encodes molecules important for the recruitment, and maturation of dendritic cells, especially follicular dendritic cells which have significant potential to cross-present tumor antigen. These include but are not limited to either of the following, used alone, or in combination with one or more additional molecule: CXCL13, CXCL12, CCL19, CCL21, soluble TGFβRI/II/III, IL-13, IL-4, IL-4R, IL-21, IL-22, IL-22R, IL-23. Molecules that are inactivated to promote antigen presentation and tumor cell uptake by APC include CD274, CD47, TGFβR, SMAD2/3, among others (See table 1).

In another embodiment of the present disclosure, the PyroTIME™ construct is expressed in normal (fibroblast or stromal cells), and/or transformed cells to generate PyroTIMECells™. These stromal fibroblast-like cells are mixed with tumor cells or tumor lysates to generate tumor specific TLS, TS-TLS in vivo. Notably, PyroTIMECells™ create a persistent anti-tumor immune response against a broad repertoire of tumor specific antigens conferring significant anti-tumor immunity

In one aspect of the disclosure, PyroTIMECells™ are developed from normal cells derived from an autologous or allogeneic setting and in another aspect from transformed cells, derived from an autologous or allogeneic setting.

The present disclosure describes the composition of chemical and methods to stimulate antigen presentation and type I and II interferon signaling in tumor cells using PyroStim™.

The present disclosure describes the composition of and methods to stimulating antigen presentation in tumor cells by expressing B2M, beta-2-macroglobulin gene using PyroStim™.

### Limited Diversity of Anti-Tumor Immune Cells

The present disclosure describes the composition of and methods to activate inflammatory tumor cell death to cause the endogenous T-cell response against antigens to be activated and diversified towards non-cross reactive epitopes selected from the same antigen (intramolecular spreading) or other antigens (intermolecular spreading) using PyroAct™.

In one aspect of the disclosure, the activation signal for PyroAct™ is functionally linked to PyroHIM™ such that the antigen encoded in PyroHIM™ stimulates an immune response which drives selective inflammatory cell death. This antigen-restricted approach to activation of pyroptosis allows for targeted activation of epitope spreading in tumors.

The present disclosure describes the composition of and methods to develop autologous or allogeneic PyroCell™ which redirect the existing immune repertoire previously generated against SARS-CoV-2 Spike S-2P and RBD antigen or influenza antigens in patients prophylactically vaccinated or those that recovered from the disease to treat cancer.

### Attenuation of Live Vaccines Negatively Impacts Antigen Presentation

The present disclosure describes the composition of and methods to develop a live cancer vaccine containing the construct, PyroKill™, which allows for the killing of any injected tumor cells that were not rejected by the immune system.

### Attenuation of Live Vaccines Negatively Impacts Cytokine Production

The present disclosure describes the composition of and methods to develop a live cancer vaccine containing the construct, PyroKill™, which allows for the killing of any injected tumor cells that were not rejected by the immune system.

### Limited Expansion of Autologous Cells

While whole cell vaccines have been demonstrated to have clinical activity, manufacturing this type of vaccine requires surgical removal of patient's tumor and ex-vivo processing of cells. It is often difficult to obtain sufficient cells for creation of the therapy and can require one to several weeks.

The present disclosure describes the composition of and methods to develop A Ilo-PyroCells™ which is composed of patient derived autologous tumor cells mixed with an allogeneic cell line (Allo) expressing PyroHIM™, PyroAct™, and PyroTIME™. Auto cells are treated with PyroStim™ prior to mixing with A Ilo-PyroCells™.

In one aspect of the disclosure, the source of the Allo-PyroCells™ would be tumor cells derived from allogeneic tumor cells, allogeneic tumor cells expanded in xenografts, normal cells including fibroblasts, macrophages, or dendritic cells, or a combination of one or more. In another aspect B2M deficient A Ilo-PyroCells™ would be created and used to avoid cross-reactivity with allogenic epitopes.

### Limited Editing Efficiency of Autologous Cells

The present disclosure describes the composition of and methods to develop Auto-Allo-PyroCell™ which is composed of patient derived autologous tumor cells (Auto) mixed with an allogeneic cell line expressing PyroHIM™, PyroAct™, and PyroTiME™.

### Elaborate Timelines for Manufacturing of Personalized Vaccines.

Vaccine manufacturing timelines are long extending out to approximately 4 months and rely on computationally derived prediction of TAA. Advanced and metastatic human cancers grow aggressively and don't have time and the power of prediction is poor.

The present disclosure describes methods to generate PyroCell™ in 2 weeks.

The present disclosure describes methods to generate A Ilo-PyroCell™ in 1 week.

### Ex-Vivo Culture Associated Modifications

The present disclosure describes the composition of and methods to develop PyroVir™ which is a viral construct expressing PyroHIM™, PyroAct™, PyroTiME™, and PyroStim™ delivered intratumorally avoid the need for ex-vivo modifications.

The two oncolytic HSV2 vectors were developed from the HG52 (6) strain. Modifications include deletion of the ICP47 and ICP34.5 genes and insertion of a GFP expression cassette expressing PyroHIM™, PyroAct™, PyroTiME™, and PyroStim™.

Patients with Advanced Cancers are Often Immunodeficient

Patients with advanced solid human cancers do not contain a robust repository of cytotoxic immune cells for various reasons including central and peripheral immune tolerance, age, immunosuppressive conditioning, chemotherapy, among others (1).

The present disclosure describes the composition of and methods to develop autologous or allogeneic PyroCell™ which redirects the existing immune repository against SARS-CoV-2 or influenza antigens to treat cancer.

In other embodiments of the present disclosure, the genetically modified cellular therapies can be administered as a stand-alone therapy, or in combination with other therapies. In one aspect, the PyroVax™ vaccine may be combined with another therapeutic agent including, but not limited to IFNγ, checkpoint inhibitors (anti-PD1, anti-CTLA-4), and adoptive T cell therapies. The present disclosure PyroCell™, can also be used in combination with monoclonal antibodies against targets including, but not limited to, PD-1, PD-L1, CTLA-4, LAG3, TIM3, TIGIT, CD40, OX40, GITR, BCL-2, or cytokines such as IL-2, IFN-γ, TNF-α, and TLR agonists.

The disclosure describes the composition of the genetic and chemical modifications used to create autologous or allogenic cellular immunotherapies, or viral immunotherapies. These immunotherapies stimulate the patient's own immune system to detect abroad repertoire of cancer specific antigens and kill heterogeneous cancer cells, conferring patients long term immunity from their cancer.

The disclosure describes PyroCells™ cellular immunotherapies which are created by genetically and chemically modifying the patient's own tumor cells. When re-introduced into the same patient PyroCells™ stimulate an anti-tumor immune response against a broad repertoire of cancer specific antigens. PyroCells™ immunotherapies are effective in a prophylactic and therapeutic setting helping control disseminated and advanced heterogeneous human cancers.

PyroVax™ DNA or mRNA encoding vectors are used to genetically modify autologous or allogeneic tumor and normal cells and turn them into cellular immunotherapies which activate a significant anti-tumor immune response against a broad repertoire of cancer specific antigens.

PyroVax™ has three components: PyroHIM™, PyroAct™, and PyroTIME™. Together, these components help break immune tolerance against tumor specific antigens and activate a polyclonal immune response against a broad repertoire of tumor specific antigens. PyroHIM™ encodes for a highly immunogenic molecule which is non-self and expressed at supra-physiological levels. PyroAct™ encodes for an activator of inflammatory cell death which triggers immunological epitope spreading and diversification of the anti-tumor T cell repertoire. PyroTIME™ encodes for molecules which remodel the immunosuppressive TIME, tumor immune microenvironment allowing cytotoxic immune cells to persist and kill tumor cells.

The following sections describe the design, manufacturing, safety, and efficacy or PyroCells™ cellular immunotherapies in pre-clinical cellular and animal models.

### Composition of PyroVax™ and PyroStim™

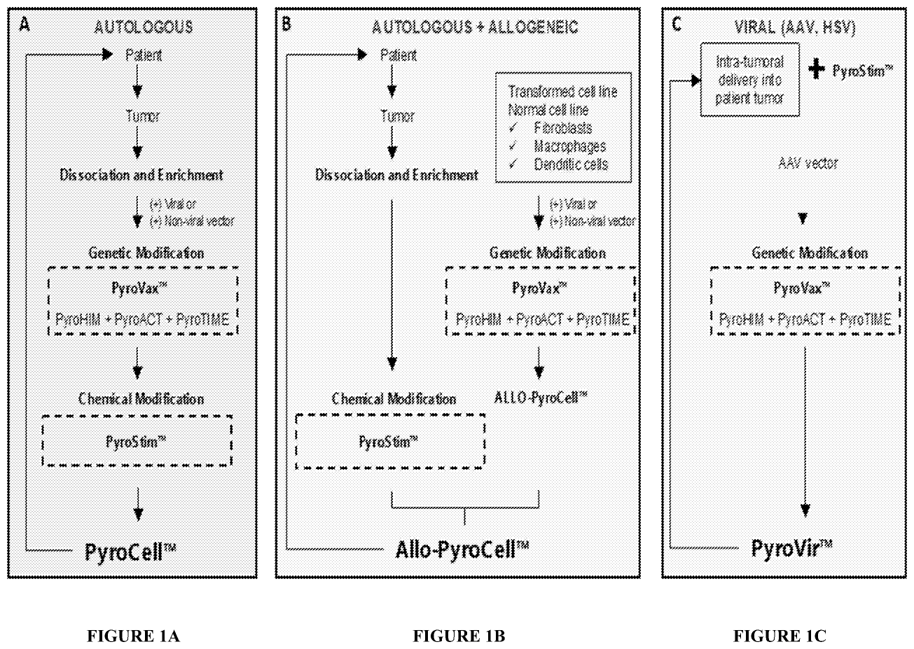

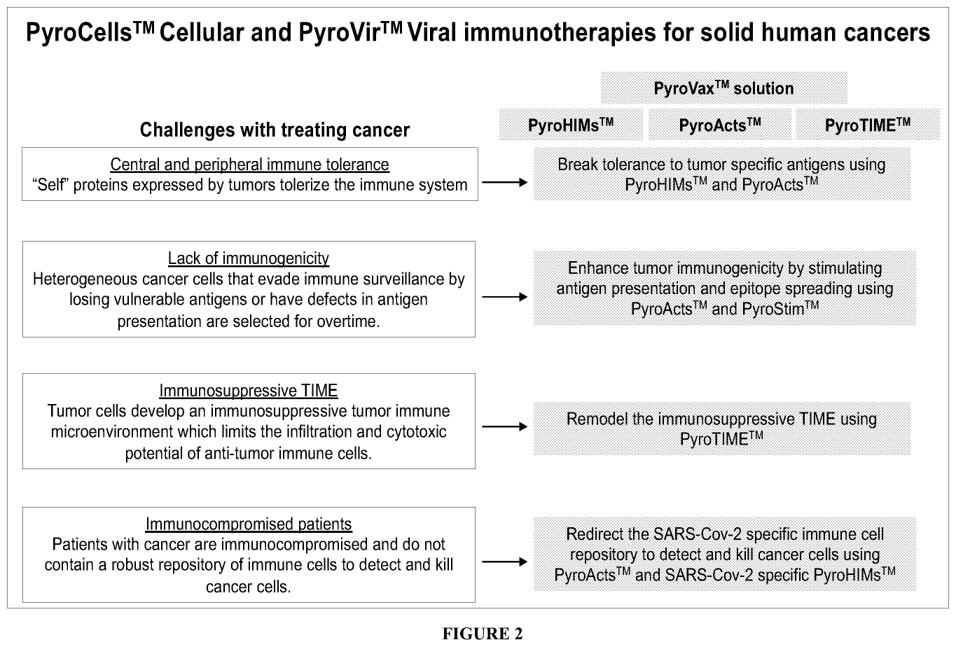

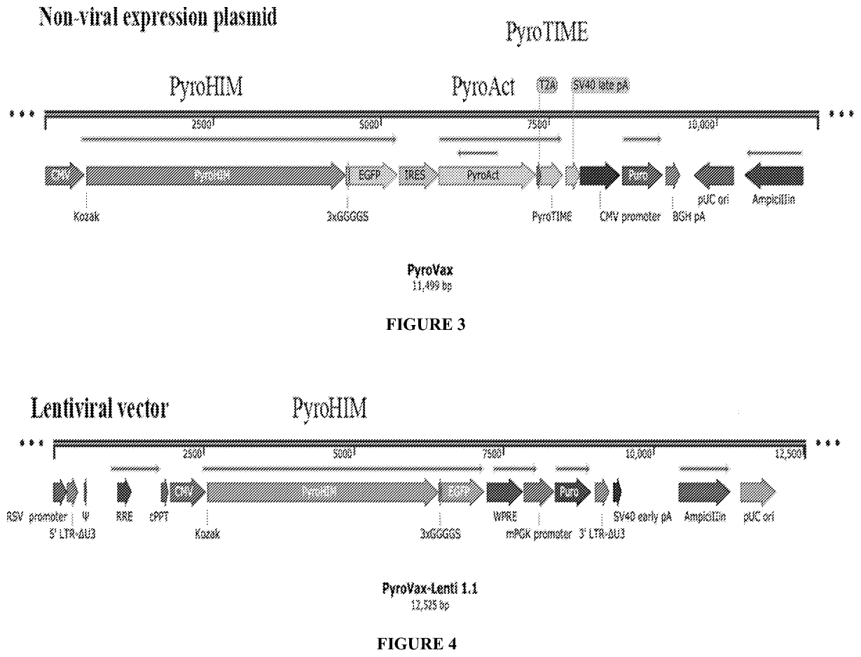

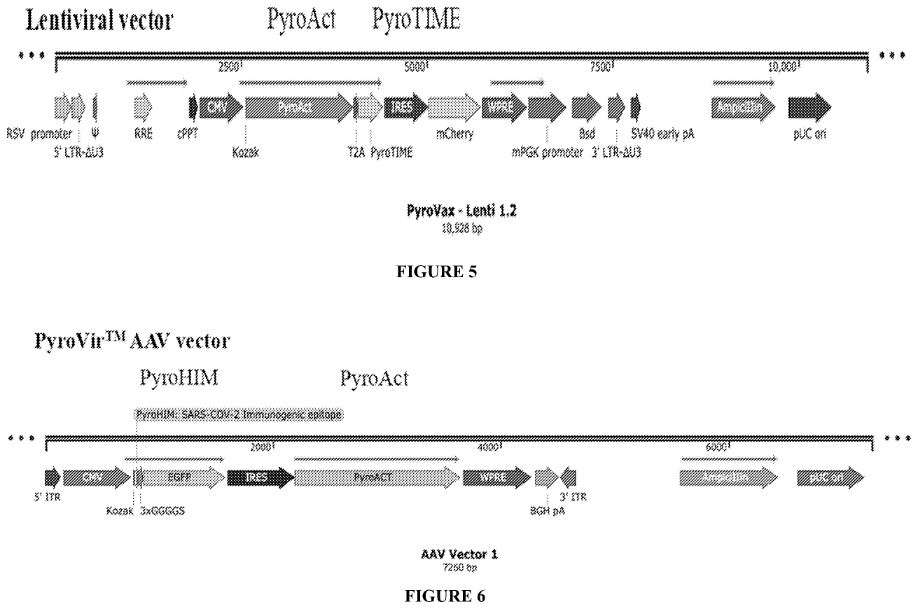

PyroVax™, is a DNA plasmid or mRNA vector(s) encoding: i) PyroHIMs™, highly immunogenic molecules, ii) PyroActs™, molecules that stimulate inflammatory cell death, and PyroTIME™, molecules that remodels the immunosuppressive TIME, and support activation, and persistence of anti-tumor immune cells. (FIG. 1A shows schematic of the PyroVax™ vector(s), table below shows examples of each of the three components).

| PyroHIM ™ | PyroACT ™ | PyroTIME ™ |
|---|---|---|
| Epitopes from SARS-Cov-2_Spike glycoprotein (S) | GSDMD | CXCL13 |
| Epitopes from SARS-Cov-2_Envelope protein (E) | GSDMD-NT | CXCL12 |
| Epitopes from SARS-Cov-2_Membrane protein (M) | AIM2 | CCL19 |
| Epitopes from SARS-Cov-2_Nucleocapsid | GSDME | CCL21 |
| phosphoprotein (N) | | |
| Epitopes from SARS-CoV-2 Spike S-2P antigen | IL33 | IL1β |
| Epitopes from SARS-CoV-2 RBD | TXNIP | CXCL9 |
| Epitopes from Enhanced Green Fluorescent Protein | IRAK1 | CXCL10 |
| Epitopes from Influenza HA antigens | NLRP3 | B2M |
| | | CD274 |
| | | CD47 |
| | | STGFβRII/III |
| | | SMAD2/3 |
| | | IL13 |
| | | IL4R |
| | | IL4 |
| | | IL22 |
| | | IL22R |
| | | LTβR |

The SARS-Cov-2 epitopes shown below are representative examples of peptide sequences of partial length or full length proteins that would be used as model antigens in humans and murine models (see table 1 and claims for others).

Human Epitopes

| SEQ | | | | |
|---|---|---|---|---|
| ID | | | | |
| NO. | IEDB # | E pitope | Antigen | Organism |
| 2 | 1309147 | YLQPRTFLL | Spike glycoprotein | SARS-CoV2 |
| 3 | 60242 | SPRWYFYYL | Nucleoprotein | SARS-CoV2 |
| 4 | 1310598 | LLLLDRLNQLESKMS | Nucleoprotein | SARS-CoV2 |
| 5 | 37473 | LLLDRLNQL | Nucleoprotein | SARS-CoV2 |
| 6 | 1310756 | QYIKWPWYI | Spike glycoprotein | SARS-CoV2 |
| 7 | 33667 | KTFPPTEPK | Nucleoprotein | SARS-CoV2 |
| 8 | 190494 | MEVTPSGTWL | Nucleoprotein | SARS-CoV2 |
| 9 | 1309115 | FTSDYYQLY | ORF3a protein | SARS-CoV2 |
| 10 | 1309137 | SIIAYTMSL | Spike glycoprotein | SARS-CoV2 |
| 11 | 16156 | FIAGLIAIV | Spike glycoprotein | SARS-CoV2 |
| 12 | 1310623 | LTDEMIAQY | Spike glycoprotein | SARS-CoV2 |
| 13 | 28050 | IPRRNVATL | Replicase polyprotein lab | SARS-CoV2 |
| 14 | 21347 | GMSRIGMEV | Nucleoprotein | SARS-CoV2 |
| 15 | 1313269 | NYNYLYRLF | Spike glycoprotein | SARS-CoV2 |
| 16 | 16737 | FLINKEMYL | Replicase polyprotein lab | SARS-CoV2 |
| 17 | 1309138 | SMWALIISV | Replicase polyprotein lab | SARS-CoV2 |
| 18 | 17385 | FPRGQGVPI | Nucleoprotein | SARS-CoV2 |
| 19 | 2998 | ALWEIQQVV | Replicase polyprotein lab | SARS-CoV2 |
| 20 | 72048 | VYIGDPAQL | Replicase polyprotein lab | SARS-CoV2 |
| 21 | 74593 | YLDAYNMMI | Replicase polyprotein lab | SARS-CoV2 |
| 22 | 1074846 | ALSKGVHFV | ORF3a protein | SARS-CoV2 |
| 23 | 1074952 | KLPDDFTGCV | Spike glycoprotein | SARS-CoV2 |
| 24 | 54680 | RLNEVAKNL | Spike glycoprotein | SARS-CoV2 |
| 25 | 69657 | VLNDILSRL | Spike glycoprotein | SARS-CoV2 |
| 26 | 1087430 | YYQLYSTQL | ORF3a protein | SARS-CoV2 |

SARS-CoV-2-specific T cell epitopes in BALB/c mice and C57BL/6 mice

| | | | | | | | |
|---|---|---|---|---|---|---|---|
| SEQ | | | | | | | |
| ID | | | | Start | End | | MHC |
| NO. | Name | Sequence | Protein | position | position | Length | restriction |

BALB/c

| | | | | | | | |
|---|---|---|---|---|---|---|---|
| 27 | N66- | FPRGQGVPINTNSSP | N | 66 | 80 | 15 | I-A^{d} |
| | 80 | | protein | | | | |
| 28 | N244- | QTVTKKSAAEASKK | N | 244 | 258 | 15 | I-A^{d} |
| | 258 | | protein | | | | |
| 29 | N351- | ILLNKHIDAYKTFPP | N | 351 | 365 | 15 | I-A^{d} |
| | 365 | | protein | | | | |
| 30 | S61- | NVTWFHAIHVSGTN | S | 61 | 75 | 15 | I-E^{d} |
| | 75 | G | protein | | | | |
| 31 | S444- | KVGGNYNYLYRLFR | S | 444 | 458 | 15 | I-A^{d} |
| | 458 | K | protein | | | | |
| 32 | ORF8 | FYSKWYIRVGARKSA | ORF8 | 41 | 55 | 15 | I-A^{d} |
| | 41-55 | | | | | | |
| 33 | S268- | GYLQPRTF | S | 268 | 275 | 8 | H2-K^{d} |
| | 275 | | protein | | | | |
| 34 | S535- | KNKCVNFNF | S | 535 | 543 | 9 | H2-D^{d} |
| | 543 | | protein | | | | |
| 35 | S1052- | FPQSAPHGV | S | 1052 | 1060 | 9 | H-2L^{d} |
| | 1060 | | protein | | | | |

C57BL/6

| | | | | | | | |
|---|---|---|---|---|---|---|---|
| SEQ ID | N9-23 | QRNAPRITFGGPSDS | N | 9 | 23 | 15 | I-A^{b} |
| NO. | | | protein | | | | |
| 36 | S62-76 | VTWFHAIHVSGTNGT | S | 62 | 76 | 15 | I-A^{b} |
| | | | protein | | | | |
| 37 | ORF3a | CYDYCIPYNSVTSSI | ORF3a | 153 | 167 | 15 | 1-A^{b} |
| | 153-167 | | | | | | |
| 38 | ORF3a | EPIYDEPTTTTSVPL | ORF3a | 266 | 280 | 15 | I-A^{b} |
| | 266-280 | | | | | | |
| 39 | ORF7a | QFAFACPDGVKHVYQ | ORF7a | 62 | 76 | 15 | I-A^{b} |
| | 62-76 | | | | | | |
| 40 | N219-226 | GFSALEPL | N | 219 | 226 | 8 | H2-K^{b} |
| | | | protein | | | | |
| 41 | M174-181 | RTLSYYKL | M | 174 | 181 | 8 | H2-K^{b} |
| | | | protein | | | | |
| 42 | S102-109 | RGWIFGTT | S | 102 | 109 | 8 | H2-K^{b} |
| | | | protein | | | | |
| 43 | S230-238 | PIGINITRF | S | 230 | 238 | 9 | H2-D^{b} |
| | | | protein | | | | |
| 44 | S263-270 | AAYYVGYL | S | 263 | 270 | 8 | H2-K^{b} |
| | | | protein | | | | |
| 45 | S471-478 | EIYQAGST | S | 471 | 478 | 8 | H2-K^{b} |
| | | | protein | | | | |
| 46 | S510-517 | VVVLSFEL | S | 510 | 517 | 8 | H2-K^{b} |
| | | | protein | | | | |
| 47 | S538-546 | CVNFNFNGL | S | 538 | 546 | 9 | H2-D^{b} |
| | | | protein | | | | |
| 48 | S820-828 | DLLFNKVTL | S | 820 | 828 | 9 | H2-D^{b} |
| | | | protein | | | | |
| 49 | ORF3a | YLYALVYFL | ORF3a | 107 | 115 | 9 | H2-D^{b} |
| | 107-115 | | | | | | |

In another aspect of the disclosure, the full-length protein or peptide derivatives of the Influenza A structural proteins, including the HA antigen (HA) and epitopes utilized for immunization, are used as antigens. Immunogenic epitopes are used alone, or in combination connected with linkers or not to enhance immunogenicity.

Identification of MHC-I restricted Cytotoxic T lymphocyte (CTL) epitopes. To predict peptides selected from SARS-Cov-2 proteins including the spike glycoprotein (S), envelope protein (E), membrane protein (M), and nucleocapsid phosphoprotein (N) that induce CTL responses the MHG-I Binding tool of Immune Epitope Database and Analysis Resource was used (IEDB; http://tools.iedb.org/mhci).

Identification of MHC-II restricted T lymphocyte epitopes. IEDB (http://www.iedb.org) was also used to predict MHC-II binding of 15-mer epitopes including proteins against human HLAs using NN-align 2.3 method. High, intermediate and low affinity epitopes were chosen.

Identification of B-cell epitope prediction. 16-mer linear B-lymphocyte (LBL) epitopes were identified using a threshold of 0.5 (ABCpreds server). The ElliPro tool of IEDB was utilized to predict linear and conformational B-cell epitopes.

Polypeptide structure validation. The ProtParam server is used to interrogate the physical and chemical properties of the construct, such as amino acid composition, molecular weight, theoretical isoelectric point (pI), grand average of hydropathicity (GRAVY), aliphatic and instability index, and half-life. The secondary structural properties of the polypeptide were analyzed using SOPMA server, modeled and refined the 3D structure model using the GalaxyWEB server. RAMPAGE server and ProSA-web tools were used to validate the refined 3D model.

### Details of Each Component of the PyroVax™ Vector (PyroHIM™, PyroAct™, and PyroTIME™).

### PyroHIM™ (Highly Immunogenic Molecules)

PyroHIM™ encodes highly immunogenic molecules. These molecules are designed to provoke a robust immune response when introduced into the patient's body. They act as antigens, triggering the immune system to recognize them as foreign and initiate an immune response. PyroHIM™ is able to make cancer cells more visible to the immune system. Cancer cells often evade the immune system by appearing similar to healthy cells. By introducing highly immunogenic molecules, PyroHIM™ helps the immune system recognize and attack cancer cells more effectively.

PyroHIM™ can include epitopes (small protein fragments) from various sources, such as SARS-CoV-2 proteins, Influenza A antigens, or other tumor-specific antigens. These epitopes are chosen because they are known to stimulate a strong immune response. By encoding these epitopes in the PyroVax™ vector, the immune system is primed to target cancer cells displaying these antigens.

### PyroAct™ (Activator of Inflammatory Cell Death):

PyroAct™ encodes molecules that trigger inflammatory cell death, a process known as pyroptosis. Pyroptosis is a form of programmed cell death that results in the release of pro-inflammatory signals, which further stimulate the immune system. PyroAct™ amplifies the immune response. When cancer cells undergo pyroptosis, they release signals that attract immune cells to the site of cell death. This not only eliminates the cancer cell but also recruits more immune cells to the tumor microenvironment. PyroAct™ encodes molecules like Gasdermin E (GSDM E) or other proteins associated with pyroptosis. When these molecules are expressed in cancer cells, they lead to pyroptosis upon exposure to specific triggers. This results in the release of danger signals, such as cytokines, which alert the immune system to the presence of cancer cells.

### PyroTIME™ (Tumor Immune Microenvironment Remodeler):

PyroTIME™ encodes molecules that remodel the tumor immune microenvironment (TIME). It works to reduce the immunosuppressive factors within the TIME and promote a pro-immune response. PyroTIME™ creates a more favorable environment for immune cells to function effectively. M any tumors create an immunosuppressive environment that hinders immune cell activity. PyroTIME™ aims to counteract this by suppressing immunosuppressive molecules and promoting the recruitment and maturation of immune cells within the tumor. PyroTIME™ encodes various molecules, including cytokines, chemokines, soluble receptor antagonists, and gene-editing tools like guide RNAs (gRNA). These components can manipulate the expression of specific genes or signaling pathways to enhance immune cell recruitment and reduce immunosuppressive factors like TGF-β (transforming growth factor-beta).

In summary, the PyroVax™ vector is a sophisticated tool for cancer immunotherapy. PyroHIM™ makes cancer cells more visible to the immune system, PyroAct™ amplifies the immune response and recruits immune cells, and PyroTIME™ remodels the tumor microenvironment to create a more immune-supportive environment. Together, these components aim to overcome the challenges associated with cancer immunotherapy by harnessing the power of the patient's immune system to target and eliminate cancer cells effectively.

### PyroHIM™: Molecular Selection

PyroHIM™ is designed to enhance the immunogenicity of the PyroCells vaccine. It serves as an adjuvant, stimulating a potent immune response against tumor cells. The choice of SARS-CoV-2-derived molecules for PyroHIM™ is grounded in several scientific principles, including high immunogenicity, broad pre-existing immunity, epitope spreading potential, molecular selection criteria, the spike protein, safety profile, and strategic synergy.

SARS-CoV-2 has demonstrated an exceptional capacity to provoke robust immune responses in infected individuals. This heightened immunogenicity is attributed to the virus's unique structure and the presence of highly antigenic epitopes, such as the spike protein. By incorporating elements from SARS-CoV-2 into PyroHIM™, the disclosure capitalizes on this intrinsic immunogenicity to amplify the immune response against tumor cells.

Due to the global impact of the COV ID-19 pandemic, a substantial proportion of the world's population has been exposed to SARS-CoV-2 either through infection or vaccination. This exposure has led to the generation of memory T cells and antibodies specific to SARS-CoV-2 antigens. The presence of pre-existing immunity in a significant portion of potential patients is strategically advantageous. PyroHIM™ acts as a beacon, drawing upon these pre-existing immune cells and redirecting them to target tumor cells, effectively repurposing the body's existing defenses for cancer immunotherapy.

SARS-CoV-2-derived molecules, including the spike protein, contain multiple immunogenic epitopes. When presented to the immune system, these epitopes can trigger epitope spreading, a process where the immune response broadens to recognize and attack a variety of antigens. The present disclosure discusses methods to exploit this phenomenon to diversify the anti-tumor immune response. As PyroHIM™ directs immune cells towards tumor cells while incorporating SARS-CoV-2 epitopes, it encourages the immune system to identify and target a broader range of tumor-specific antigens. This strategy mitigates the risk of tumor escape due to antigenic variation, a common challenge in cancer immunotherapy.

The specific molecules chosen from SARS-CoV-2 for PyroHIM™ are selected based on their immunogenicity, safety, and strategic fit for cancer immunotherapy:

The spike protein of SARS-CoV-2 is a key antigenic target of the immune response during natural infection and vaccination. Its prominent role in eliciting strong immune reactions makes it an ideal choice for PyroHIM™. Furthermore, the spike protein's widespread recognition by the immune system enhances the likelihood of recruiting memory T cells and antibodies to target tumor cells.

The selected SARS-CoV-2 molecules have a well-characterized safety profile from extensive research during the COVID-19 pandemic. This safety record is crucial, ensuring that PyroHIM™ does not introduce unexpected adverse effects or immune reactions.

The choice of SARS-CoV-2 molecules aligns strategically with the disclosure's broader goals. By repurposing the immune response developed against SARS-CoV-2 for cancer treatment, the present disclosure describes methods and compounds to increase the utility of existing immunity, making PyroCells an innovative and efficient therapy.

PyroHIM™'s scientific rationale for selecting specific molecules, particularly those related to SARS-CoV-2, is rooted in their proven immunogenicity, safety, and potential to induce epitope spreading. This unique approach leverages the global experience with SARS-CoV-2 to advance the field of cancer immunotherapy, offering a promising solution to address the unmet needs of cancer patients.

PyroHIM™ was designed using several strategies and with multiple pathways in mind, including selective immune cell recruitment, antigen presentation enhancement, T cell activation and expansion, epitope spreading, and inflammatory cell death (pyroptosis).PyroHIM™ is designed to attract and recruit specific immune cell populations to the tumor site. This recruitment is facilitated by the inclusion of SARS-CoV-2-derived epitopes in PyroHIM™, which act as molecular “baits” to draw immune cells. These epitopes have a twofold purpose: they serve as targets for pre-existing memory T cells generated against SARS-CoV-2, and they stimulate the maturation and activation of antigen-presenting cells (APCs) at the tumor site.

As PyroHIM™ recruits immune cells, it also promotes the presentation of tumor-specific antigens by APCs. The presence of SARS-CoV-2 epitopes within PyroHIM™ triggers APCs to engulf tumor cells and present a diverse array of tumor antigens, including patient-specific neoantigens. This process, called cross-presentation, is critical for the activation of cytotoxic T cells that can target and eliminate tumor cells.

Upon encountering the tumor-derived antigens presented by APCs, T cells are activated. PyroHIM™ enhances this activation by providing a rich source of co-stimulatory signals and cytokines, such as GM-CSF (Granulocyte-Macrophage Colony-Stimulating Factor). GM-CSF further stimulates the maturation of dendritic cells and promotes the expansion and activation of effector T cells, specifically cytotoxic CD8⁺ T cells.

PyroAct™'s incorporation of SARS-CoV-2 epitopes, known to be highly immunogenic, induces epitope spreading. This phenomenon broadens the immune response beyond the initially targeted tumor-specific antigens. As a result, the immune system becomes attuned to a wider spectrum of tumor antigens, reducing the risk of tumor immune escape due to antigenic variation.

One of the unique features of the approach described in the present disclosure is the induction of pyroptosis in the PyroCells. Pyroptosis is a highly inflammatory form of cell death that releases a myriad of cellular contents, including additional tumor antigens and inflammatory signals. This event amplifies the immune response, drawing in more immune cells and intensifying the anti-tumor effect.

### PyroTIME™: Remodeling the Tumor Immune Microenvironment

Through modification of the microenvironment, the present disclosure discusses enhancing anti-tumor immune responses. Such modifications include immunosuppressive microenvironment disruption, reprogramming of immune cells, enhanced antigen presentation, and anti-angiogenic effects.

PyroTIME™ is strategically designed to disrupt the immunosuppressive microenvironment that often surrounds tumors. It accomplishes this through the targeted release of cytokines and chemokines, such as IFN-γ (Interferon-gamma) and CXCL 9/10. These signaling molecules attract immune cells, particularly cytotoxic T cells and natural killer (NK) cells, into the tumor microenvironment.

PyroTIME™ doesn't merely attract immune cells; it also reprograms them to adopt a more aggressive anti-tumor phenotype. IFN-γ, released by PyroTIME™, enhances the cytotoxic activity of T cells and NK cells, making them more effective at recognizing and eliminating tumor cells. Additionally, it can polarize tumor-associated macrophages (TAMs) from an M2 immunosuppressive phenotype to an M1 pro-inflammatory phenotype, further aiding in tumor clearance.

PyroTIME™ complements the antigen presentation process initiated by PyroHIM™ By creating an inflamed tumor microenvironment, it encourages APCs to be more efficient at engulfing tumor material, processing it, and presenting it to T cells. This synergy between PyroHIM™ and PyroTIME™ leads to a robust and sustained anti-tumor immune response.

PyroTIME™ also exerts anti-angiogenic effects by inhibiting the recruitment of new blood vessels to the tumor site. This action limits the nutrient supply to the tumor and creates a hostile environment for its growth and survival.

PyroHIM™ and PyroTIME™ work synergistically to enhance tumor immunogenicity and remodel the Tumor Immune Microenvironment (TIME). PyroHIM™ recruits and activates immune cells, induces epitope spreading, and amplifies the immune response, while PyroTIME™ disrupts the immunosuppressive TIME, reprograms immune cells for a more aggressive anti-tumor function, and enhances antigen presentation. This multifaceted approach holds immense promise for revolutionizing cancer immunotherapy by making the TIME more permissive to anti-tumor immune responses and ensuring sustained tumor control.

### Interactions Between PyroHIM™ and PyroTIME™

The present disclosure discusses use of various techniques and cells in order to enhance anti-tumor immune responses, including immune cell recruitment and activation, remodeling the tumor microenvironment, reducing immunosuppressive cells, increasing inflammatory cytokines, enhancing antigen presentation, and the synergistic effect of modification of multiple techniques and materials.

PyroHIM™ is designed to recruit and activate immune cells, including dendritic cells, T cells, and natural killer (NK) cells, within the Tumor Immune Microenvironment (TIME). These immune cells are crucial for recognizing and targeting tumor cells.

PyroTIME™ complements PyroHIM™ by altering the Tumor Microenvironment (TME) to be more immunogenic. It can potentially reduce immunosuppressive factors and cells within the TME. This remodeling includes:

PyroTIME™ may reduce the presence of regulatory T cells (Tregs) and myeloid-derived suppressor cells (M DSCs), which are known to suppress anti-tumor immune responses. This reduction helps create a more permissive immune environment.

PyroTIME™ can increase the production of pro-inflammatory cytokines (e.g., IL-2, IFN-γ) within the TME. These cytokines stimulate effector T cells and enhance their cytotoxic activity.

By promoting the maturation of dendritic cells and increasing the expression of major histocompatibility complex (MHC) molecules, PyroTIME™ improves antigen presentation. This allows for better recognition of tumor antigens by T cells.

PyroHIM™ and PyroTIME™ act synergistically to create an immune-favorable TME. PyroHIM™ recruits immune cells, while PyroTIME™ helps create an environment that activates these recruited cells. This combined effect amplifies the anti-tumor immune response, potentially overcoming the immunosuppression typically seen in the TME.

### Interactions Between PyroHIM™, PyroTIME™, and PyroStim™:

Further techniques and materials described in the present disclosure enhance anti-tumor immune responses, immune cell activation, antigen presentation, immune checkpoint blockade, epitope spreading, and precision and personalization of treatments.

PyroHIM™ and PyroTIME™ both contribute to immune cell activation. PyroHIM™ recruits immune cells and promotes their infiltration into the tumor site, while PyroTIME™ remodels the TME to enhance immune cell activation. PyroStim™ further boosts this activation through the inclusion of immune checkpoint inhibitors and immunostimulatory cytokines.

PyroHIM™ enhances antigen presentation by promoting the maturation of dendritic cells. PyroStim™ complements this by including tumor-specific antigens, ensuring a diverse array of tumor antigens are presented to immune cells. This combination enhances the immune system's recognition of tumor cells.

PyroStim™ contains immune checkpoint inhibitors, such as anti-PD-L1 antibodies, which block the PD-L1/PD-1 interaction. This prevents T cell exhaustion and enhances their cytotoxic activity. When used in conjunction with PyroHIM™ and PyroTIME™, the immune checkpoint blockade is applied in an environment where immune cells are actively engaged, maximizing its effectiveness.

PyroHIM™ and PyroTIME™ potentially promote epitope spreading by creating conditions where dying tumor cells release a variety of tumor antigens. PyroStim™ enhances this process by boosting the immune response against these released antigens. This synergy can lead to a broader and more potent anti-tumor immune response.

All three components can be personalized based on the subject's tumor profile. This ensures that the treatment is tailored to the unique characteristics of the subject's cancer, maximizing its efficacy.

In summary, PyroHIM™, PyroTIME™, and PyroStim™ work in a coordinated manner to create a highly immunogenic and pro-inflammatory tumor microenvironment. They recruit immune cells, remodel the immune-suppressive elements within the tumor, activate the immune response, and enhance antigen presentation. This comprehensive approach addresses various aspects of the anti-tumor immune response, potentially leading to a more effective and durable therapeutic effect.

### Technology of PyroCells™

The present disclosure provides mechanisms, techniques and materials to redirect immune response, modify immunological memory, increase function of epitope cross-reactivity, activate immune cells, and modify supraphysiological expression.

Redirecting the immune response is one aspect of the present disclosure, initially generated against SARS-CoV-2, towards the targeted elimination of cancer cells. This redirection is achieved through the PyroCell™ immunotherapy system and the PyroVax™ vector, which have been meticulously designed to exploit the immunological memory and response elicited by SARS-CoV-2 exposure or vaccination. This section elaborates on the mechanism, rationale, and preliminary evidence supporting this redirection.

The immune system's memory is a key component of this redirection. Patients who have been exposed to SARS-CoV-2, recovered from the infection, or have been vaccinated against it, harbor a reservoir of immune cells with specificity towards SARS-CoV-2 antigens, including CD8⁺ T cells, CD4⁺ T cells, and B cells. These memory cells can be harnessed for recognizing cancer-specific antigens.

The PyroVax™ vector incorporates highly immunogenic epitopes from SARS-CoV-2 antigens, such as the spike glycoprotein (S), envelope protein (E), membrane protein (M), nucleocapsid phosphoprotein (N), as well as other immunodominant antigens. These epitopes have been strategically selected based on their propensity for cross-reactivity with cancer-specific antigens.

PyroVax™ includes PyroHIMs™, which encode highly immunogenic molecules. These molecules serve as potent activation signals for immune cells, triggering their effector functions, including cytokine release and cytotoxic activity. By linking PyroHIMs™ to SARS-CoV-2-derived epitopes, PyroVax™ ensures the activation of memory immune cells with specificity for SARS-CoV-2 antigens.

In some embodiments, PyroHIMs™ are linked to reporter molecules, enabling supraphysiological expression, detection, and enrichment of PyroCells™ expressing the PyroVax™ vector. This facilitates the selective expansion of PyroCells™ with high immunogenicity, which are then targeted against cancer cells. Effectiveness of Immune Response Redirection. Preclinical Models Preliminary evidence from preclinical studies using PyroCell™ and PyroVax™ in animal models has demonstrated: Efficacy: Substantial regression of established tumors in mice. Diversity of Immune Response: Activation and diversification of the T-cell repertoire against a range of tumor-specific antigens. Long-Term Immunity: Durable protection against cancer recurrence.

### Pyroptotic Mechanisms in PyroCells

M any mechanisms are involved in the activity of PyroCells, including mechanisms such as T cell activation: granzyme B release, gasdermin E activation, pore formation, inflammatory response, and cell lysis. Possible challenges and side effects include the inflammatory response, off-target effects, and immunosuppression, while mitigation strategies include personalization, dose optimization, monitoring, and combination therapies.

PyroCells are designed to stimulate a robust immune response against cancer cells. T cells, a critical component of the immune system, play a central role in this process. Upon activation, cytotoxic T cells, in particular, release molecules such as granzyme B.

Granzyme B is a protease enzyme released by cytotoxic T cells. It is a key mediator of the immune response against infected or cancerous cells. Granzyme B can enter target cells, including cancer cells.

Inside the cancer cells targeted by PyroCells, granzyme B initiates a cascade of events that ultimately lead to the activation of gasdermin E. Gasdermin E is a critical protein in pyroptosis; it forms pores in the cell membrane.

Gasdermin E pores created in the cell membrane disrupt its integrity. This results in the release of cellular contents, including pro-inflammatory cytokines and danger-associated molecular patterns (DAMPs).

The release of pro-inflammatory cytokines and DAMPs signals neighboring immune cells, particularly macrophages and dendritic cells, to mount an immune response. This response includes the recruitment of more immune cells to the site and the activation of an adaptive immune response against the cancer cells.

The disruption of the cell membrane integrity by gasdermin E pores leads to cell lysis, or cell bursting. These releases additional pro-inflammatory molecules and cellular debris.

While the pyroptotic mechanism is a powerful tool in cancer immunotherapy, there are potential challenges and side effects to consider: The hallmark of pyroptosis is the rapid and robust inflammatory response. While this is desired for the immune system to recognize and attack cancer cells, excessive inflammation can lead to adverse effects, including flu-like symptoms, fever, and, in severe cases, cytokine storms. Granzyme B, while highly specific for target cells like cancer cells, can potentially enter and activate gasdermin E in unintended cells. This off-target effect could result in damage to healthy tissues and organs. In some cases, the inflammatory response may be counteracted by regulatory immune mechanisms, leading to immunosuppression. This can hinder the overall effectiveness of the treatment.

To address these challenges and minimize potential side effects associated with pyroptotic mechanisms, the present disclosure describes several strategies: PyroCells are personalized for each patient, targeting their specific cancer cells. This approach minimizes off-target effects, as the vaccine is tailored to the individual's unique cancer profile. Careful dose optimization is performed to balance a strong immune response with safety. This involves determining the appropriate amount of PyroCells to minimize excessive inflammation while ensuring efficacy. Rigorous monitoring of patients during clinical trials allows for the early detection of adverse effects, enabling timely intervention and adjustment of treatment plans. The present disclosure explores combination therapies, including immunosuppressive agents or anti-inflammatory drugs, to modulate the immune response and mitigate potential side effects.

In summary, the pyroptotic mechanism involving T cell-secreted granzyme B and gasdermin E is one aspect of PyroCells' therapeutic action against cancer. While there are challenges and potential side effects associated with this mechanism, personalized treatment, dose optimization, vigilant monitoring, and strategic combination therapies help to mitigate these risks. These efforts aim to increase the benefits of pyroptosis while ensuring patient safety and treatment effectiveness.

### Allogeneic Mechanism of Action

The mechanism of action of the proposed therapy, which involves the use of allogenic cells, includes a complex but highly orchestrated process that harnesses the power of the immune system to target and eliminate cancer cells.

Enhanced Antigen Presentation: PyroCells™ are designed to enhance antigen presentation within the tumor microenvironment. This is achieved through multiple mechanisms:

Upregulation of MHC Class I Molecules: PyroCells™ increase the expression of Major Histocompatibility Complex (MHC) Class I molecules on the surface of tumor cells. MHC Class I molecules play a critical role in presenting tumor antigens to CD8⁺ T cells.

Activation of Dendritic Cells: PyroCells™ stimulate the maturation and activation of dendritic cells within the tumor microenvironment. Dendritic cells are professional antigen-presenting cells that capture, process, and present tumor antigens to CD8⁺ T cells, initiating an immune response.

CD8⁺ T Cell Activation: The C D 8⁺ T cells recruited to the tumor site encounter the tumor-specific antigens presented by MHC Class I molecules on PyroCells™ and dendritic cells. This interaction activates the CD8⁺ T cells, turning them into cytotoxic effector T cells.

Tumor Cell Killing: Activated CD8⁺ T cells recognize and specifically target tumor cells expressing the presented antigens. They release cytotoxic molecules, such as perforin and granzymes, to induce apoptosis (cell death) in the tumor cells.

Epitope Spreading: As tumor cells are killed, they release additional tumor antigens into the tumor microenvironment. This process, known as epitope spreading, diversifies the pool of antigens available for immune recognition. CD8⁺ T cells can recognize and target a broader range of tumor epitopes, reducing the risk of tumor escape due to antigenic variation.

Elaboration on PyroStim™: Composition, Mechanism of Action, Advantages

PyroStim™ is a carefully composed therapeutic composition with the potential to significantly enhance the anti-tumor immune response. Its multi-faceted mechanism of action, including immune checkpoint blockade, cytokine-mediated enhancement, and antigen presentation, makes it a promising addition to Pyrojas' arsenal in the fight against cancer. Its advantages include enhanced specificity, synergy with other therapies, and the ability to overcome immune evasion strategies employed by tumors.

Composition: PyroStim™ is a proprietary composition designed to enhance the effectiveness of Pyrojas' cancer immunotherapy. It is composed of a carefully selected combination of molecules and factors that collectively boost the anti-tumor immune response. The composition includes:

Immune Checkpoint Inhibitors: PyroStim™ incorporates immune checkpoint inhibitors (ICIs) such as anti-PD-L1 (Programmed Death-Ligand 1) antibodies. These ICIs block the PD-L1/PD-1 interaction, preventing the tumor from evading the immune system by suppressing T cell activity.

Cytokines: PyroStim™ contains specific cytokines, including interleukins (e.g., IL-2 and IL-12), interferons (e.g., IFN-γ), and TN F alpha family members known for their roles in enhancing immune responses. These cytokines stimulate the activation and proliferation of cytotoxic T cells, natural killer (NK) cells, and other immune effector cells.

Mechanism of Action: PyroStim™ operates through a multi-faceted mechanism to potentiate the immune response against the tumor:

Immune Checkpoint Blockade: The inclusion of immune checkpoint inhibitors in PyroStim™, such as anti-PD-L1 antibodies, plays a central role. By blocking the PD-L1/PD-1 interaction, PyroStim™ prevents the inhibition of T cell activity, allowing activated T cells to target and eliminate tumor cells more effectively.

Cytokine-Mediated Enhancement: Cytokines within PyroStim™, such as IL-2, IL-12, and IFN-γ, act as potent immunostimulants. They activate immune effector cells, particularly cytotoxic T cells and NK cells, which are critical for tumor cell recognition and destruction.

Antigen Presentation: Inclusion of tumor-specific antigens in PyroStim™ enhances antigen presentation. This helps in directing the immune response toward tumor-specific targets, further increasing the specificity of the anti-tumor immune response.

Synergy with PyroHIM™ and PyroTIME™: PyroStim™ synergizes with PyroHIM™ and PyroTIME™, which recruit and activate immune cells and remodel the Tumor Immune Microenvironment (TIME). The combined effect amplifies the anti-tumor response, potentially overcoming immunosuppression within the tumor and promoting durable tumor control.

### Advantages of PyroStim™

Enhanced Immune Response: PyroStim™ acts as a powerful immune booster. By blocking immune checkpoints and providing immunostimulatory cytokines, it ensures that the immune system is primed and ready to attack the tumor.

Increased Specificity: The inclusion of tumor-specific antigens in PyroStim™ enhances the specificity of the immune response. This reduces the risk of off-target effects and ensures that the immune system primarily targets tumor cells.

Synergy with Existing Therapies: PyroStim™ is designed to work synergistically with Pyrojas' other therapeutic components, including PyroHIM™ and PyroTIME™. This combination approach increases the chances of a robust and sustained anti-tumor response.

Overcoming Immune Evasion: Immune checkpoint inhibitors within PyroStim™ counteract mechanisms used by tumors to evade the immune system. This can potentially make tumors more vulnerable to immune attack.

### Methodology for Creating PyroCell™

### Isolation of Target Cells

Techniques and Equipment: Target cells are cultured in a controlled environment using aseptic techniques, including a laminar flow hood, CO2 incubator, and sterile culture vessels. The choice of specific target cell lines is based on the intended application and tumor type. Authentication is performed using short tandem repeat (STR) profiling. Potential Challenges and Optimizations: Ensuring the purity of the target cell line is crucial. Regular STR profiling and mycoplasma testing are conducted to maintain cell line integrity. Strict aseptic procedures are followed to prevent contamination during cell culture.

Transfection with PyroVax™ Vector (DNA or mRNA)

Techniques and Equipment: Transfection Reagents: ipofection or electroporation based on cell type. Commercial transfection reagents or custom-made formulations are employed. PyroVax™ Vector Preparation: The PyroVax™ vector is purified and quantified using established molecular biology techniques. Potential Challenges and Optimizations: Transfection Efficiency: Optimization of transfection conditions (e.g., voltage, reagent concentration) is crucial to increase vector uptake by target cells. Vector Quality Control: Rigorous quality control of the PyroVax™ vector ensures consistent results.

### Selection and Expansion of Transfected Cells

Techniques and Equipment: Selection Markers: Cells are often transfected with a selectable marker gene, e.g., antibiotic resistance or fluorescent proteins. Cell Expansion: Transfected cells are expanded in culture to obtain enough for downstream applications. Potential Challenges and Optimizations: Selection Pressure: Optimizing the concentration of selection agents is necessary to balance cell survival and marker expression. Cell Growth Optimization: Media composition, seeding density, and passage intervals are modified to promote cell growth while maintaining transgene expression.

### PyroCell™ Production

Techniques and Equipment: Cell Harvest: Transfected cells are harvested at the desired stage of growth. Quality Control: Cell viability, transgene expression, and other relevant parameters are assessed. Potential Challenges and Optimizations: Cell Viability: Careful timing of cell harvest is critical to increase cell viability. Consistency: Batch-to-batch consistency is maintained through rigorous quality control measures.

### Cryopreservation

Techniques and Equipment: Cryoprotectants: Cells are cryopreserved using validated cryoprotectant solutions. Cryopreservation Containers: Vials or cryobags are used for storage.

Potential Challenges and Optimizations: Cell Recovery: Optimizing cryoprotectant composition and freezing rates ensures high cell recovery upon thawing. Storage Conditions: Strict storage conditions (−80 °C or liquid nitrogen) are maintained for long-term preservation.

### Manufacturing of the Therapeutic Cellular Immunotherapies.

To manufacture autologous PyroCells™, surgically resected primary and/or metastatic patient tumors are dissociated into single cells using a cell dissociation buffer which prevents the disruption of antigen-loaded MHC-I and MHC-II molecules on the cell surface. Tumor cells are enriched by removing stromal cells, including immune cells, endothelial cells, and fibroblasts, using antibody-mediated negative selection. The PyroVax™, composed of either DNA vector or mRNA construct encoding the components of PyroHIM™, PyroAct™ and PyroTIME™, is introduced into tumor cells using viral transduction or electroporation. PyroVax™ expressing cells, termed PyroCells™, are chemically enriched and treated with PyroStim™, a cocktail of cytokines and chemokines which increase tumor antigen presentation, and the gene expression levels of ISGs, interferon-stimulated genes. After comprehensive gene-editing, QA/QC, and in-vitro functional validation studies, GM P compliant immunotherapeutic tumor cells are expanded to achieve therapeutic doses (0.1, 0.5, 1×10\hat{ }6), rendered replication incompetent, frozen into individual doses, and shipped to the physician for the treatment of the patient. The entire manufacturing process is G M P compliant and is completed within two weeks.

To manufacture A Ilo-PyroCell™: Briefly, patient-derived autologous tumor cells are chemically modified using PyroStim™ and mixed with allo-PyroCell™, an allogeneic cell line modified with either a DNA vector or mRNA construct encoding the components of PyroVax™, which includes sequences of PyroHIM™, PyroAct™, and PyroTIME™ (at either of the ratios: 1:1, 1:2, 1:5, 1:10, 1:100). This mixture is then shipped to the physician for the treatment of the patient. The entire manufacturing process is G M P compliant and is completed within two weeks. As compared to autologous approaches, allogeneic cellular therapies have abundantly available TAAs, are standardizable, scalable, less variable, and cost-effective. Cell lines can be developed from solid or liquid cancers, metastatic or circulating cancer cells, and can be derived from one or multiple individuals.

To manufacture PyroVir™ to deliver PyroVax™ intratumorally. The present disclosure describes the composition of and methods to generate PyroVir™, an AAV, HSV1/2, or other cancer specific virus, expressing the PyroVax™ vector. When administered intratumorally, the virus infects tumor cells and stimulate an immune response against PyroHIMs™ and a broad repertoire of cancer specific antigens. PyroVir™ overcomes the various challenges associated with ex vivo modifications of autologous or allogeneic cells.

### Functional Validation of the PyroVax™ Construct Used to Make PyroCells™ or PyroVir™.

Validation of the PyroVax™ vector. PyroVir™ AAV virus, lentivirus, or a non-viral expression plasmid is used to deliver the PyroVax™ construct into normal and transformed human and murine cells. After chemical selection and flow cytometry-based enrichment, the transcript and protein expression levels of PyroVax™ components: the PyroHIMs™, PyroActs™, and PyroTIME™ are quantified using RT-PCR, and immunoblotting or flow cytometry. Efficient delivery of PyroVax™ into normal or transformed cells is confirmed using immunoblotting, flow cytometry, and ELISA based studies.

### PyroCells™ Cellular and PyroVir™ Viral Immunotherapies Induce Pyroptotic Tumor Cell Death.

Next, the impact of the expression of PyroVax™ in normal and transformed human and murine cells is functionally validated. The expression of an activator of the inflammasome and pyroptotic cell death like GSDM E, GSDM D, AIM2 innate immune sensor, or cytokine IL33 among others (see table 1 and claims) when linked to a PyroHIM™ promote immunity against diverse tumor antigens by activating immunological epitope spreading and diversifying the anti-tumor T cell repertoire.

The PyroAct™ molecule serves as a trigger for the inflammatory cell death of PyroCells™ when killed by PyroHIM™ antigen-specific T cells. Therefore, the frequency of PyroCells™ or PyroVir™ infected cells that died by apoptosis (Annexin V⁺) versus pyroptosis (PI⁺) when co-cultured with cytotoxic CD8⁺ T cells that recognize the PyroHIM™ antigen is quantified. PyroAct™ containing PyroCells™ or PyroVir™ exclusively undergo pyroptotic cell death over apoptotic cell death. Consistently, inhibitors of pyroptosis limit the impact of PyroAct™ whereas inhibitors of other forms of cell death have no significant impact. Collectively, these data demonstrate that a combination of a PyroHIM™ and a PyroAct™ triggers preferential pyroptotic cell death over apoptotic cell death.

To demonstrate that PyroAct™ induces epitope spreading from the immunodominant PyroHIM™ epitope to less dominant cancer antigens, PyroCells™ or PyroAct™ deficient PyroCells™ are co-cultured with PyroHIM™ antigen specific-CTLs in the presence of antigen presenting cells (APC), dendritic cells or macrophages. Both PyroCells™ and PyroAct™ deficient PyroCells™ are killed by PyroHIM™ antigen specific-CTLs, although with noteworthy differences. First, while PyroAct™ deficient PyroCells™ die predominantly via Caspase-1 mediated apoptosis, PyroAct™ expressing PyroCells™ die preferentially via pyroptosis. These observations demonstrate that PyroCells™ are killed via inflammasome or Gasdermin E mediated pyroptotic cell death.

To evaluate whether the observed pyroptotic cell death stimulates immunological epitope spreading, the reactivity of CTLs retrieved from the tumor-CTL-APC co-cultures is examined compared to previously unreactive cancer specific antigens using co-culture assays and ELISPOT cytokine release assay. CTLs derived from the co-cultures containing PyroCells™ kill the parental PyroVax™ deficient tumor cells but CTLs derived from PyroAct™ deficient PyroCells™ have no reactivity towards parental cells. Collectively, these data demonstrate that PyroCell™ cellular immunotherapies are sensitive to killing by CTLs and that PyroAct™ triggers pyroptotic inflammatory tumor cell death.

LDH enzyme release is used to monitor pyroptosis (7), among other markers including cytokine released because of inflammasome activation: IL-1β and IL-1RA. Activation of the inflammasome induces the formation of Gasdermin-D pores on the cell membrane, causing IL-1β and IL-18 secretion, and the influx of water molecules leading to cell swelling and subsequent rupture (pyroptosis).

Interestingly, SARS-Cov-2 antigens themselves are shown to have a significant intrinsic potential to activate the inflammasome and pyroptosis. PyroHIM S™ encoding the SARS-CoV-2 proteins E, M, and specific ORFs induced efflux of K⁺ ions creating an imbalance which results in oxidative stress, damage to the mitochondria, and activation of the NLRP3 inflammasome (8, 9). Interestingly, ORF3a and ORF8b activate the inflammasome directly (8, 9).

### Immunization and Validation of Mice Against Anti-SARS-Cov-2

Construction, expression, and purification of recombinant spike protein. The code optimized recombinant DNA encoding the Spike protein expressed by the SARS-Cov-2 virus was introduced into a mammalian gene expression lentiviral vector (pLV[Exp]-Puro-CMV) vector (Vector Builder, USA). The DNA sequence of the assembled vector was verified by DNA sequencing. The sequence of the inserts and vectors are provided.

Female BALB/c mice are subcutaneously immunized with two doses of Spike protein or saline control. High levels of SARS-CoV-2-specific IgG antibodies and neutralization antibodies are elicited in of the immunized mice at 2 weeks after boost immunization. Immunized mice are intranasally challenged with mouse-adapted strain at passage 6, MASCp6 (1.6×10⁴ PFU), and lung tissues are collected for virological and histopathological analysis at 5 days after challenge, as described previously (10). All the PBS-treated mice sustain high amounts of viral RNA loads in the lung at 5 days after challenge. By contrast, immunized mice demonstrate a significant reduction in viral RNA loads. Immunofluorescence staining for SARS-CoV-2 S protein is used to detect viral proteins in the lungs of immunized mice versus PBS-immunized mice. No pathological damage in the lungs is observed in immunized mice, whereas inflammatory lung injury, with focal perivascular and peribronchiolar inflammation, as well as thickened alveolar septa are present in the lungs of the control mice.

ELISA. ELISA is performed to detect SARS-CoV-2-specific IgG antibodies. Briefly, ELISA plates are precoated with SARS-CoV-2 Spike protein (1 pg/ml) overnight at 4 °C and blocked with 2% milk in PBST for 2 h at 37 °C. Serially diluted sera are added to the plates and incubated for 2 h at 37 °C. After four washes, the bound antibodies are detected by incubation with horseradish peroxidase (HRP)-conjugated anti-mouse IgG antibody (Thermo Fisher, USA, 1:5000) for 1 h at 37 °C. The reaction is visualized by addition of substrate 3,3′,5,5′-Tetramethylbenzidine (TMB) (Sigma, USA) and stopped by adding H2SO4 (1N). The absorbance at 450 nm is measured by an ELISA plate reader.

SARS-CoV-2 neutralization assay. A micro-neutralization assay is carried out to detect neutralizing antibodies against SARS-CoV-2 as previously described (11, 12). Briefly, mouse sera at 2-fold serial dilutions are incubated with SARS-CoV-2 (100 TCID50) for 1 h at 37 °C and added to Vero cells. The cells are observed daily for the presence or absence of virus-induced cytopathic 9 effects (CPE) and recorded at 3 dpi. Neutralizing antibody titers are expressed as the reciprocal of the highest dilution of serum that completely prevent CPE in Vero cells.

### PyroCells™ and PyroVir™ Viral Immunotherapies are Safe and Tolerable.

Mice immunized against SARS-Cov-2 are treated with the PyroCells™ cellular or PyroVir™ viral immunotherapy and monitored for acute safety concerns and long-term adverse outcomes. Mice maintain their weight and show no signs of morbidity throughout the course of the treatment and on long-term follow up. Gross and histological assessment of all major organs systems by a pathologist is performed to evaluate any signs of toxicity or unintended adverse outcomes including auto-immune reactions. The impact of combining the therapeutic vaccines with the current standard of care (chemotherapy) or with immune-checkpoint inhibitors is also be tested.

### PyroCells™ Cellular and PyroVir™ Viral Immunotherapies Control Disease Burden and Improve Survival Outcomes.

Next, the anti-tumor efficacy of the immunotherapy is determined using murine models of solid (TNBC, melanoma, ovarian cancer) and hematological (leukemias, lymphomas, and myelomas) human cancers. The MTD determined previously is utilized to test the anti-tumor efficacy of PyroCells™ immunotherapies in vivo.

Treatment of mice bearing established disease with PyroCells™ immunotherapy alone, or in combination with immune checkpoint inhibitors control the disease burden and increase the overall survival of mice. Further, re-challenge of complete responders with parental tumor result in complete rejection of the tumor highlighting persistence of a persistent anti-tumor immunological memory response. Notably, when used in a prophylactic setting, the allo-PyroCells™ immunotherapies inhibit the emergence and establishment of disease burden by parent tumor cells not expressing PyroHIMs™.

Immune evasion by tumor cells is driven by a loss of antigen presentation molecules like MHC-I or B2M. Therefore, the impact of vaccines on tumors which are predominantly B2M or MHC-I deficient is evaluated in vivo. Interestingly, PyroCells™ render MHC-I or B2M deficient cancer cells sensitive to killing by CD8⁺ T cells whereas PyroAct™ extend the sensitivity to diverse additional tumor epitopes in vivo. These findings demonstrate activation of antigen independent killing.

PyroCells™ cellular and PyroVir™ viral immunotherapies redirect the anti-SARS-Cov-2 immune response against a broad repertoire of cancer specific antigens.

A comprehensive evaluation of the intra-tumoral immune infiltrate is performed. A significant increase in the infiltration of antigen specific cytotoxic CD8⁺ T cells, CD4⁺ T cells, and B cells in tumors derived from mice treated with the PyroCells™ cellular immunotherapy occurs as compared to control counterparts. Notably, combining PyroCells™ immunotherapy with immune checkpoint inhibitors significantly enhances the intra-tumoral infiltration and persistence of anti-tumor cytotoxic T cells.

Patients with cancer are immunocompromised due to various reasons (1) and do not have an available robust repository of immune cells to control aggressive solid human cancers. However, patients with cancer who have recovered from a SARS-Cov-2 infection or have been vaccinated with any of the approved vaccines against SARS-Cov-2, contain a robust pool of SARS-Cov-2 antigen specific immune cells. The disclosure described here re-directs or repurposes the immune response against SARS-CoV-2 towards killing cancer cells.

Highly immunogenic SARS-Cov-2 epitopes are utilized as PyroHIM S™ operably linked to activators of inflammatory cell death, to re-direct the patient's pre-existing anti-viral immune response against SARS-Cov-2 (see table 1 containing examples of PyroHIM S™) towards cancer specific antigens. ELISPOT and tetramer-based flow cytometry are used to confirm the presence of PyroHIM™ antigen specific CD4⁺ and CD8⁺ T cells, SARS-Cov-2 specific T cells, and notably several novel TAA-specific CD8⁺ and CD4⁺ T cells in mice. Paired scRNA-seq and TCRa/b sequencing are used to test for clonal expansions of polyvalent T cells against a broad repertoire of cancer specific antigens besides the immunodominant PyroHIM™ epitope. Immunized mice develop cross-primed T cells to known cancer antigens and importantly to newly arising TAAs, tumor-associated antigens. Transcriptomics analysis of tumors is used to demonstrate a significant decrease in the expression of target antigens and/or TAAs in tumors derived from mice that received the PyroCells™ immunotherapy as compared to control counterparts. No adverse auto-immune reactions occur in humans or mice.

To evaluate whether the observed pyroptotic cell death stimulates immunological epitope spreading, antigen reactivity is examined of CTLs retrieved from the spleens of mice that received the vaccination or not, compared to previously unreactive cancer specific antigens using T cell and tumor cell co-culture killing assays and ELISPOT cytokine release assay. CTLs derived from mice treated with PyroCells™ kill the parental PyroVax™ deficient tumor cells but CTLs derived from PyroAct™ deficient PyroCells™ have no reactivity towards parental cells. These data demonstrate that mice treated with PyroCells™ develop antigen specific T cells which recognize not only the immunodominant PyroHIM™ antigen, but notably various other tumor-associated antigens.

Treatment of mice bearing established disease with PyroCells™ immunotherapy alone, or in combination with immune checkpoint inhibitors control the disease burden and increase the overall survival of mice. Importantly, re-challenge of complete responders with parental tumor cells not containing the epitope results in complete rejection of the parental tumor highlighting the diversification or spreading of the epitope and the presence of an immunological memory response. Notably, when used in a prophylactic setting, the allo-PyroCells™ immunotherapies inhibit the emergence and establishment of disease burden by parent tumor cells not expressing PyroHIMS™.

Immune evasion by tumor cells is driven by a loss of antigen presentation molecules like MHC-I or B2M. Therefore, the impact of vaccines on tumors which are predominantly B2M or MHC-I deficient is evaluated in vivo. Interestingly, PyroCells™ render MHC-I or B2M deficient cancer cells sensitive to killing by CD8⁺ T cells whereas PyroAct™ extend the sensitivity to diverse additional tumor epitopes in vivo. These findings demonstrate activation of antigen independent killing.

### PyroTherapies™ Redirect the Anti-SARS-Cov-2 Immune Response Against Uterine Leiomyomas

The safety and efficacy of using PyroTherapies™ for the treatment of benign tumors, including uterine leiomyomas or fibroids, termed FibroTherapy™, is tested. The immunocompetent Eker rat model of spontaneous uterine fibroids and an immunodeficient murine model of human uterine fibroid xenografts are used. Briefly, immunocompetent Eker rat with spontaneous uterine fibroids or immunocompromised Balb/c mice with transplanted human patient derived uterine fibroid xenografts are prophylactically vaccinated against an immunogen. As a proof of concept, the M-spike protein expressed by the SARS-Cov-2 virus is used as a model immunogen derived from PyroHIMs™, others could be used as well. All rats develop a robust humoral and adaptive immune response against the immunizing epitope several weeks after immunization. Next, a luciferase tagged episomal expression vector (EEV) or viral vector containing the DNA encoding the immunogen used for prophylactic immunization is directly injected into the fibroids. For the Eker model, intra-fibroid injection is performed laparoscopically under MRI-guidance, whereas for the human PDX model direct injection is used because the transplant is accessible. Bioluminescent imaging is used to confirm fibroid specific expression of the DNA vector in both models. Next, antigen specific cell therapy, either TCR-T cells, or CAR-T/NK cells targeting the immunogenic antigen is administered. Several weeks later, complete immunological rejection of the rat uterine fibroids and human uterine fibroid xenografts, without any adverse effects, is observed in all animals. Notably, while prophylactic immunization alone is sufficient at inducing a robust anti-fibroid CD4⁺ and CD8⁺ T cell response in Eker rats, antigen specific allogeneic T or NK cell therapy eliminated large established fibroids. These pre-clinical safety and efficacy data rationalize the advancement of FibroTherapy™ to human clinical trials.

Presented below are examples discussing the cells, proteins and nucleic acid sequences contemplated for the discussed applications. The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

### Example 1

### PyroCells Vaccines Data

### Expression and Characterization of Indicated Proteins in Human Breast Cancer Cells.

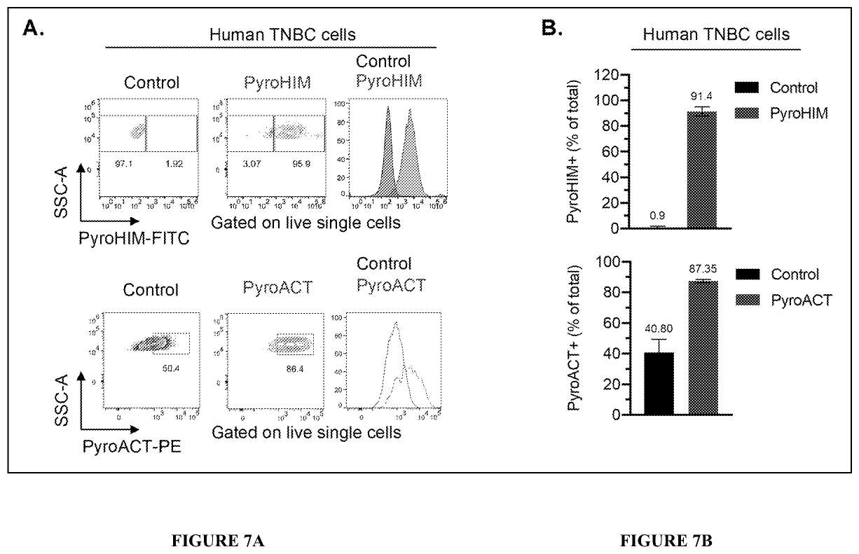

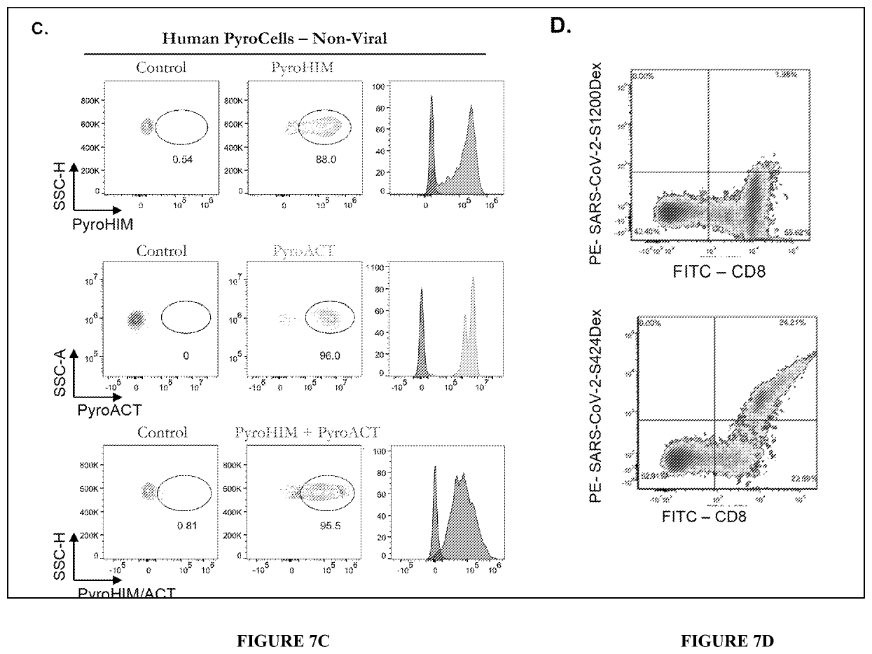

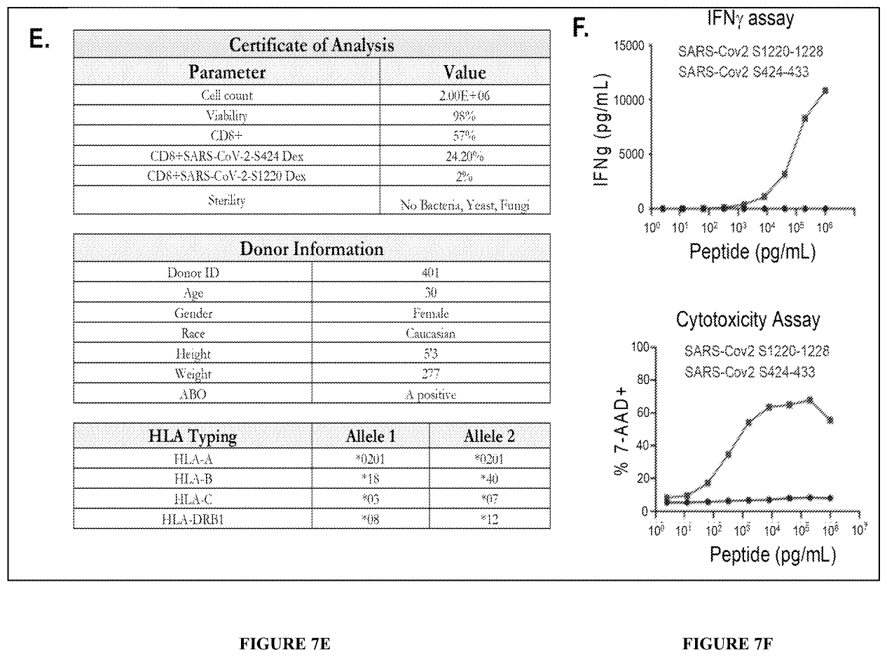

Lentiviral Vector Transduction and Protein Expression: Using lentiviral vectors, successful transduction of human TNBC cells with the genes of interest is achieved. Contour plots (FIG. 7A) demonstrate distinct expression levels of the indicated proteins in these cells post-transduction. A comprehensive quantification of this data (FIG. 7B) emphasized the efficiency of the lentiviral transduction process, showcasing significant protein expression in the transduced cells as compared to control cells. Support: Demonstrating efficient transduction and protein expression in human TNBC cells reinforces the feasibility of the proposed approach. Successful expression in a challenging cell line like TNBC vouches for the efficacy and adaptability of the proposed constructs and transduction methods.

Non-viral Approach for Protein Expression: Exploring alternative methodologies, a non-viral approach was also employed to achieve protein expression in breast cancer cells. Contour plots representing this data (FIG. 7C) displayed comparable expression levels to the lentiviral method, suggesting the potential versatility of constructs for diverse delivery mechanisms. Support: Showcasing protein expression achieved via non-viral means offers a less risky, potentially safer, and versatile alternative for therapeutic delivery. This widens the therapeutic window and enhances adaptability, making the project more appealing for funding.

### Example 2

### Tumor Burden Analysis in Murine Models

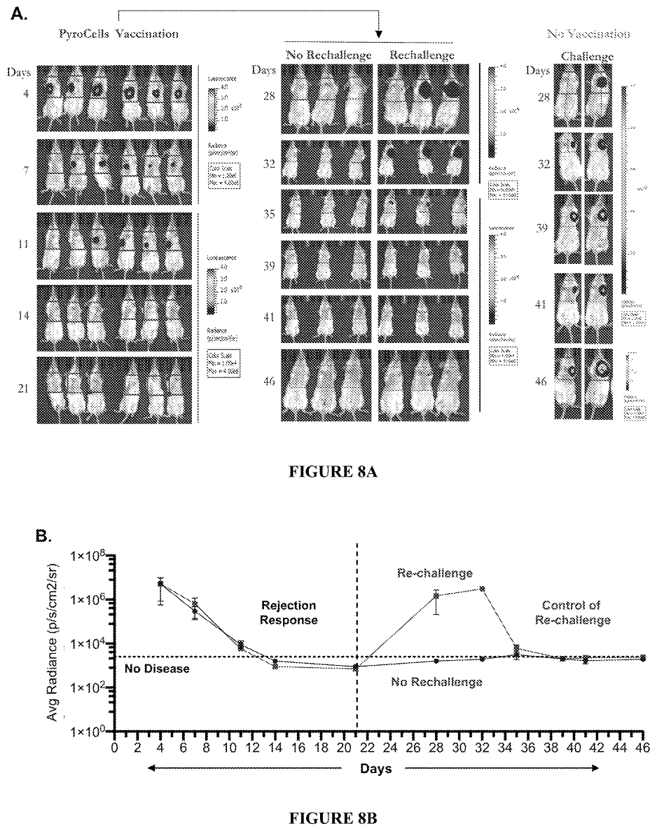

Initial Assessment of Tumor Burden: In murine models, a comprehensive assessment of tumor burden was conducted. Both average radiance and total flux were measured, providing key metrics to understand tumor progression. These metrics were presented as both bar graphs (FIG. 8A) and line charts (FIG. 8B), allowing for clear visual representation and easy interpretation of the data over time. Support: Presenting tangible evidence of tumor burden metrics (both average radiance and total flux) in murine models validates the real-world applicability of the proposed therapy. Effective reduction in tumor burden provides a strong case for the potential clinical efficacy of the constructs.

Rechallenge Experiments: To understand the protective capabilities of therapeutic constructs, mice bearing specific tumor burdens were imaged at indicated time intervals (FIG. 8A). After a span of 21 days, a subset of these mice underwent a rechallenge with the parental tumor. Interestingly, the data (FIG. 8B) showcased a substantial protective effect in mice previously immunized with the vaccine, indicating its potential not only in reducing tumor burden but also in providing long-term protective effects against cancer recurrence. Support: The rechallenge experiments underscore the long-term benefits and potential protective capabilities of the therapeutic constructs. By demonstrating resistance against cancer recurrence, emphasizing not just treatment but potential prevention aspects.

### Example 3

### Extended Tumor Burden Analysis

Long-Term Implications and Rechallenge Experiments: A detailed time-course study was performed where mice bearing tumors were imaged at specific intervals (FIG. 8C). Post the 21-day mark, the mice were either rechallenged with the parental tumor or left unchallenged. The data reinforced the earlier findings, emphasizing the consistent therapeutic potential of the constructs in controlling cancer growth and potentially preventing recurrence upon rechallenge. The results provide a comprehensive account of the in vitro and in vivo efficacy of the therapeutic constructs. From effective protein expression in human TNBC cells to significant reduction in tumor burden in murine models, the findings pave the way for potential clinical applications of these constructs in cancer therapy. Support: The extended time-course study further endorses the long-term therapeutic potential of the constructs. Showing control over cancer growth and recurrence prevention in an extended setting makes a compelling case for this application, as it promises long-term patient benefits.

### Example 4

### PyroTIMER CAR-T Cells Data

PyroTIMER: TGFβRI-II fusion protein,
SEQ ID NO: 50
atggaggcggcggtcgctgctccgcgtccccggctgctcctcctcgtgctggcggcggcggcggcggcggc
ggcggcgctgctcccgggggcgacggcgttacagtgtttctgccacctctgtacaaaagacaattttacttgtgt
gacagatgggctctgctttgtctctgtcacagagaccacagacaaagttatacacaacagcatgtgtatagctga
aattgacttaattcctcgagataggccgtttgtatgtgcaccctcttcaaaaactgggtctgtgactacaacatattg
ctgcaatcaggaccattgcaataaaatagaacttccaactactggccctttttcagtaaagtcatcacctggccttg
gtcctgtgacgatcccaccgcacgttcagaagtcggatgtggaaatggaggcccagaaagatgaaatcatctg
ccccagctgtaataggactgcccatccactgagacatattaataacgacatgatagtcactgacaacaacggtgc
agtcaagtttccacaactgtgtaaattttgtgatgtgagattttccacctgtgacaaccagaaatcctgcatgagca
actgcagcatcacctccatctgtgagaagccacaggaagtctgtgtggctgtatggagaaagaatgacgagaa
cataacactagagacagtttgccatgaccccaagctcccctaccatgactttattctggaagatgctgcttctccaa
agtgcattatgaaggaaaaaaaaaagcctggtgagactttcttcatgtgttcctgtagctctgatgagtgcaatgac
aacatcatcttctcagaagaatataacaccagcaatcctgacgacaaaactcacacatgcccaccgtgcccagc
acctgaactcctggggggaccgtcagtcttcctcttccccccaaaacccaaggacaccctcatgatctcccgga
cccctgaggtcacatgcgtggtggtggacgtgagccacgaagaccctgaggtcaagttcaactggtacgtgga
cggcgtggaggtgcataatgccaagacaaagccgcgggaggagcagtacaacagcacgtaccgtgtggtca
gcgtcctcaccgtcctgcaccaggactggctgaatggcaaggagtacaagtgcaaggtctccaacaaagccct
cccagcccccatcgagaaaaccatctccaaagccaaagggcagccccgagaaccacaggtgtacaccctgc
ccccatcccgggaggagatgaccaagaaccaggtcagcctgacctgcctggtcaaaggcttctatcccagcg
acatcgccgtggagtgggagagcaatgggcagccggagaacaactacaagaccacgcctcccgtgctggac
tccgacggctccttcttcctctacagcaagctcaccgtggacaagagcaggtggcagcaggggaacgtcttctc
atgctccgtgatgcacgaggctctgcacaaccactacacgcagaagagcctctccctgtctccgggtaaa

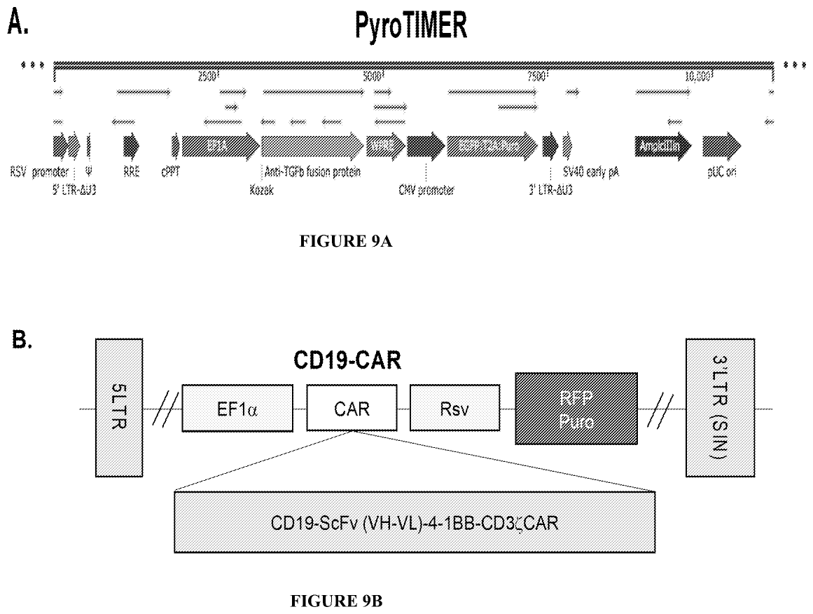

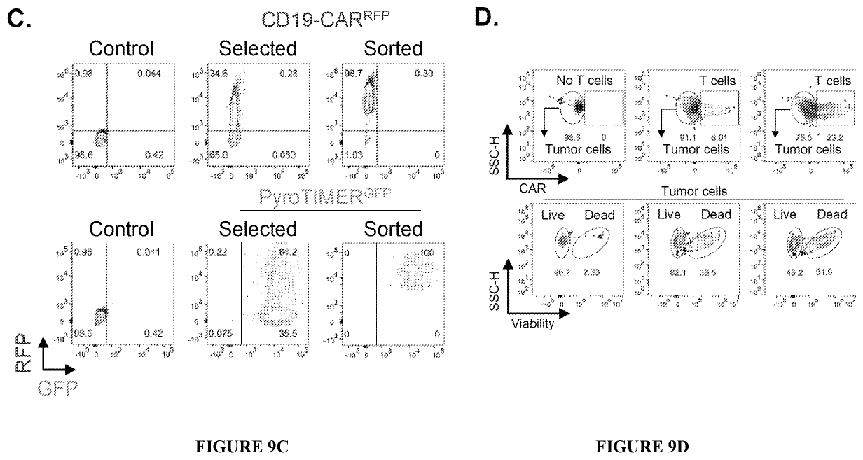

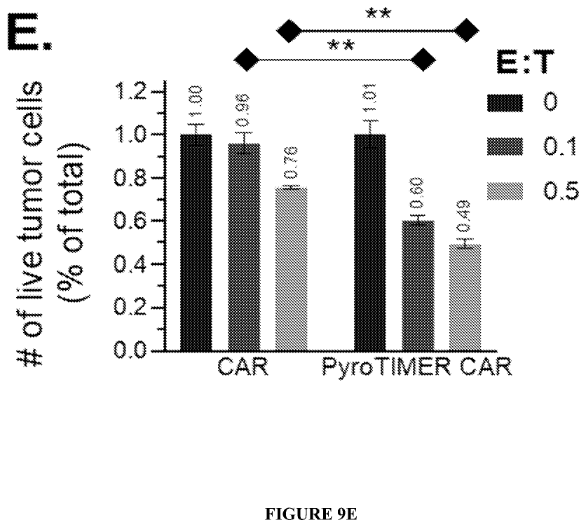

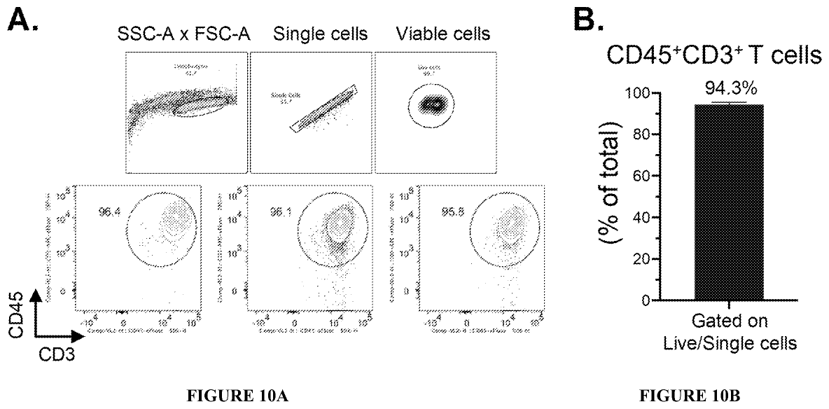

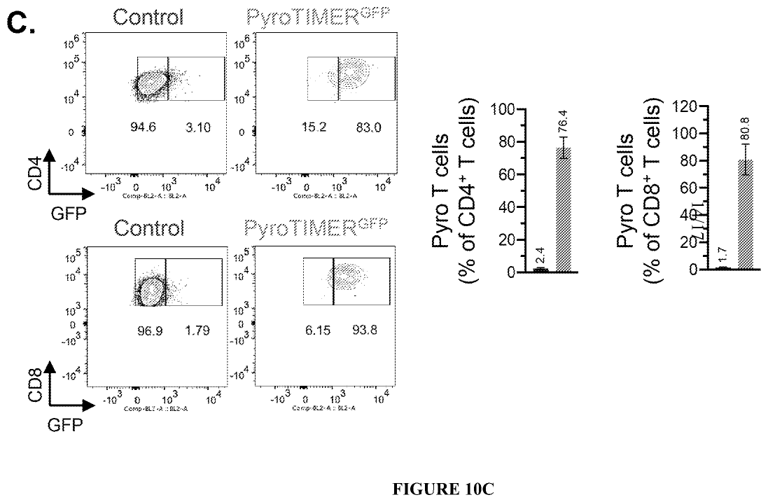

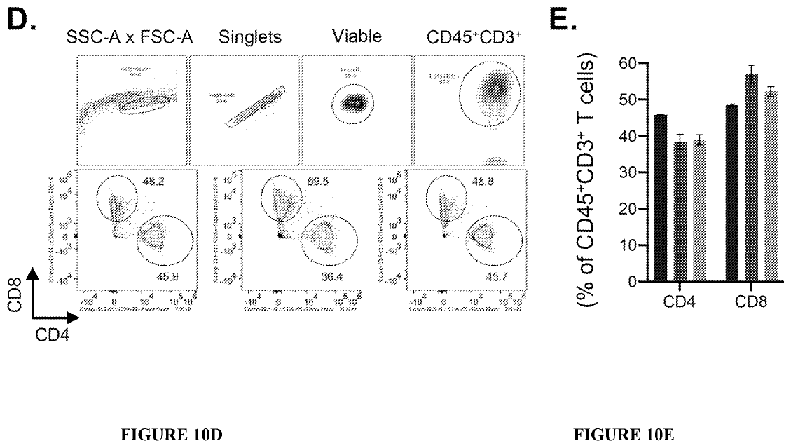

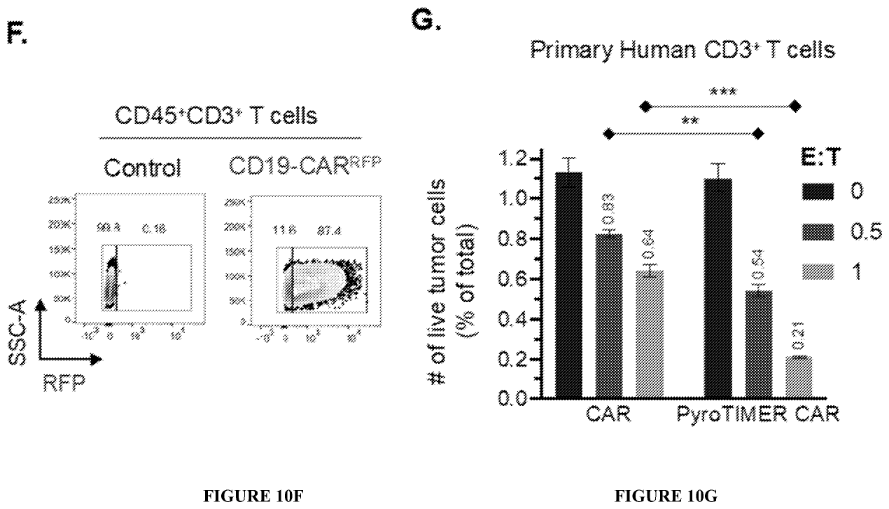

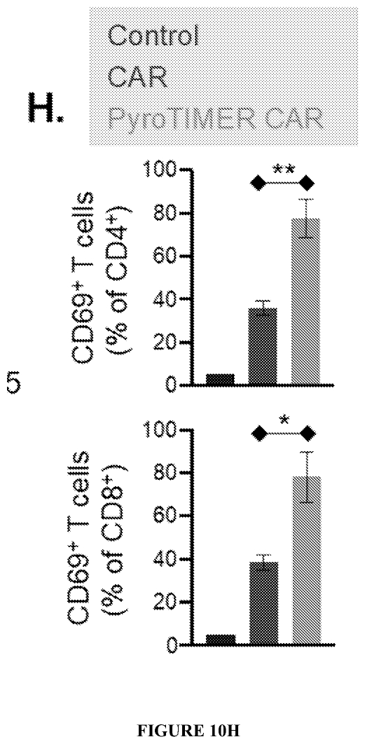

PyroTIMERs, a new class of synthetic proteins to remodel the tumor immune microenvironment and promote CAR-T cell anti-tumor activity. Biochemical and functional characterization of the lead PyroTIMER, a recombinant fusion protein including the ectodomains of TGFβRI and TGFβRII, demonstrated its sustained ability to inhibit all isoforms of TGF-β. The promising results suggest PyroTiMERs as effective therapeutic agents for enhancing CAR-T cell therapy, requiring further research to improve the design and evaluate safety and efficacy in preclinical models. In a series of preliminary studies, PyroTIMER CD19-CAR T cells were generated using JurkatT cells (FIG. 9A, FIG. 9B and FIG. 9C) and peripheral human blood-derived CD3+ T lymphocytes (FIG. 10A through FIG. 10D). The CD3+ T lymphocytes were enriched, activated using CD3/CD28 Dynabeads, and expanded in IL-2 media. Following transduction with the PyroTIMER construct and a second-generation anti-human CD19 CAR, single- and double-positive cells were selected and enriched using chemical selection and flow-based cell sorting to achieve stable expression of both the PyroTIMER and the CAR over time (FIG. 9C, FIG. 10C and FIG. 10F). Proper controls, including non-transfected cells and cells transfected with a non-functional version of PyroTiMER, are utilized to rule out non-specific effects.

To evaluate the efficacy of PyroTIMER CD19 CAR-T cells, a co-culture killing assay was performed using CD19 antigen expressing Raji lymphoma cells in the presence of 100 pM TGF-β1. PyroTIMER CD19 CAR-T cells or CD19 CAR-T cells were co-cultured with the Raji cells at increasing effector to target ratios (FIG. 9D, FIG. 9E and FIG. 10G). Results showed that PyroTIMER CD19 CAR-T cells remained activated and exhibited significantly greater killing of target cells compared to CD19 CAR-T cells alone at all tested ratios (FIG. 9D, FIG. 9E, FIG. 10G). Notably, FIG. 10G demonstrates the superior cytotoxic prowess of PyroTIMER CD19 CAR-T cells, and FIG. 10H accentuates the heightened activation, evident from the surge in CD69+ activated T cells.

Predominance in TGF-β Rich Environments: The data underscores the PyroTiMER CAR-T cells' unparalleled performance, particularly in TGF-β rich milieus. The co-culture killing assays with CD19-expressing Raji lymphoma cells, presented in FIG. 9D, FIG. 9E, FIG. 10E, and FIG. 10F, were executed in the presence of 100 pM TGF-β1, replicating the immunosuppressive ambiance of tumors overflowing with TGF-β. The superior killing efficacy of PyroTIMER CD19 CAR-T cells in comparison to traditional CD19 CAR-T cells in this environment offers incontrovertible evidence of their enhanced functionality in settings abundant in TGF-β. These finding indicate that PyroTIMER technology improves the cytotoxicity of CD19 CAR-T cells against CD19-expressing cells in TGF-β rich microenvironments. Notably, inhibiting the TGF-β pathway in hematological malignancies using CAR-T cells is particularly crucial since TGF-β plays a critical role in creating an immunosuppressive TIME in CD19 malignancies.

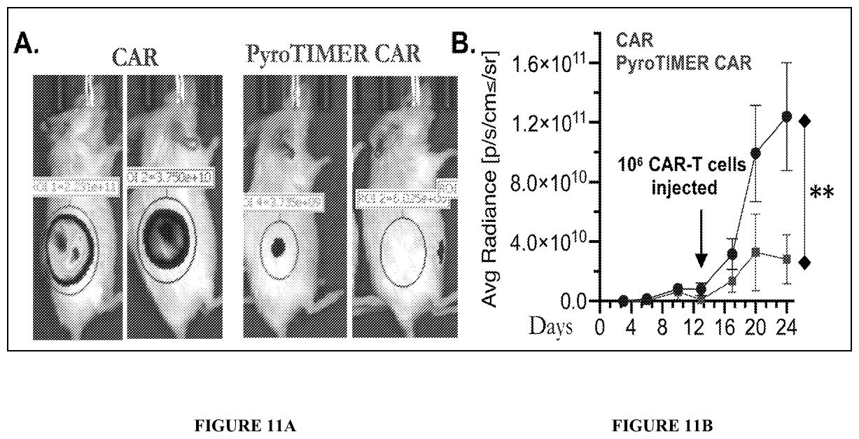

In Vivo Efficacy Validation: Notably, PyroTiMER CAR-T cells were significantly more effective at controlling CD19+ Raji lymphoma disease burden in NOD.Cg-PrkdcscidIL2Rgtm1Wjl/Sz mice (n=4, per group) as compared to traditional CD19 CAR-T cells (FIG. 11). The NOD.Cg-PrkdcscidIL2Rgtm1Wjl/Sz mouse model, depicted in FIG. 11, was employed to bring the laboratory findings into a real-world context. The superior efficacy of PyroTIMER CAR-T cells in controlling CD19+ Raji lymphoma, as compared to their traditional counterparts, not only reinforces their heightened therapeutic potential but also underscores the translational feasibility of the technology in pragmatic therapeutic settings.

Drawing upon the amassed data, it becomes evident that PyroTIMER CAR-T cells not only epitomize a novel approach to CAR-T cell therapy but also offer robust evidence in support of the present disclosure. By effectively sidelining the suppressive impacts of TGF-β in the tumor microenvironment, PyroTIMER CAR-T cells signal a transformative step in cancer immunotherapy, ensuring that claim 22 stands on solid scientific and technical ground, poised for both clinical application and commercial success.

### Example 5

### Choice of Uterine Leiomyomas (Fibroids) as a Model:

1. Prevalence and Clinical Significance: Uterine leiomyomas, commonly referred to as fibroids, are noncancerous growths of the uterus that often occur in women of reproductive age. They are highly prevalent, with estimates suggesting that up to 70-80% of women may develop fibroids during their lifetime. Fibroids can vary in size, number, and location within the uterus, leading to a range of clinical symptoms, including heavy menstrual bleeding, pelvic pain, and reproductive issues. Due to their prevalence and clinical significance, fibroids represent a substantial burden on women's health and healthcare systems.

2. Limited Treatment Options: Traditional treatments for fibroids include medication, surgery (myomectomy or hysterectomy), and minimally invasive procedures. However, these treatments are not always suitable for all subjects, and they may come with significant side effects, risks, or long recovery times. Additionally, they do not address the underlying cause of fibroid development.

3. Unmet Medical Need: Given the limitations of current treatments and the high prevalence of fibroids, there is a significant unmet medical need for effective, minimally invasive, and targeted therapies that can specifically shrink or eliminate fibroids while preserving the uterus. FibroTherapy™ aims to address this unmet need.

### Implications and Extensions to Other Benign Tumors:

While FibroTherapy™ was initially developed for uterine leiomyomas (fibroids), its underlying principles and mechanisms of action hold promise for potential extensions to other benign tumors. Here are some potential implications and extensions:

Tissue-Specific Targeting: The key to FibroTherapy™ is its ability to selectively target and remodel fibrotic tissue within the uterus. This tissue-specific targeting is achieved through the use of PyroCells™, which are genetically engineered to express specific molecules that interact with fibrotic tissue. This targeting strategy could potentially be adapted to other benign tumors that involve fibrosis or excessive extracellular matrix deposition. Conditions such as keloids, hypertrophic scars, and desmoid tumors, which are characterized by fibrotic tissue growth, might benefit from a similar approach.

Customization: FibroTherapy™ demonstrates the potential for personalized medicine in the treatment of benign tumors. By engineering PyroCells™ to express molecules tailored to the specific characteristics of the target tissue, it's possible to customize the therapy for different benign tumor types. This approach allows for a high degree of specificity and precision in treatment.

Minimally Invasive Nature: FibroTherapy™ is designed to be delivered through minimally invasive techniques, such as laparoscopy or hysteroscopy. This minimizes the need for open surgery and reduces the associated risks and recovery times. The minimally invasive nature of this approach can be advantageous for the treatment of various benign tumors located in accessible anatomical sites.

Potential Safety Profile: Since FibroTherapy™ relies on the targeted action of PyroCells™ within the tumor microenvironment, it may have a favorable safety profile compared to more systemic treatments. This aspect could be explored in the context of other benign tumors, where minimizing off-target effects is crucial.

Clinical Research: To explore the extension of FibroTherapy™ to other benign tumors, further research and clinical trials would be necessary. This research could involve adapting the therapy to the specific characteristics of different tumor types and evaluating its safety and efficacy in diverse patient populations.

In summary, while FibroTherapy™ was initially developed for uterine leiomyomas (fibroids), its tissue-specific targeting and minimally invasive nature open possibilities for potential extensions to other benign tumors characterized by fibrosis or similar pathological features. These extensions could offer new treatment options for subjects with a range of benign tumor conditions, addressing unmet medical needs in multiple clinical settings.

As described herein, the present disclosure provides compositions and methods for the treatment of cancer. In certain aspects, the cancer includes a form of acute lymphoblastic leukemia (ALL), acute myeloid leukemia (AML), adrenocortical carcinoma, aids-related cancer, Kaposi sarcoma, AIDS-related lymphoma, primary CNS lymphoma, anal cancer, astrocytoma, atypical teratoid/rhabdoid tumor, central nervous system cancer, basal cell carcinoma of the skin, skin cancer, bile duct cancer, bladder cancer, bone cancer (includes Ewing sarcoma, osteosarcoma and malignant fibrous histiocytoma), brain tumor, breast cancer, bronchial tumors, non-Hodgkin lymphoma (including Burkitt lymphoma), carcinoid tumor, carcinoma of unknown primary origin, cardiac tumor, medulloblastoma and other CNS embryonal tumor, germ cell tumor, cervical cancer, childhood cancer, bile duct cancer (including cholangiocarcinoma), chordoma, (bone cancer), chronic lymphocytic leukemia (CLL), chronic myelogenous leukemia (CM L), chronic myeloproliferative neoplasms, colorectal cancer, craniopharyngioma, lymphoma, cutaneous t-cell lymphoma, mycosis Fungoides and Sezary syndrome, breast cancer, ductal carcinoma in situ (DCIS), embryonal tumors, medulloblastoma, endometrial cancer, ependymoma, esophageal cancer, esthesioneuroblastoma, Ewing sarcoma, extracranial germ cell tumor, extragonadal germ cell tumor, eye cancer, intraocular melanoma, retinoblastoma, fallopian tube cancer, gallbladder cancer, gastric cancer, gastrointestinal carcinoid tumor, gastrointestinal stromal tumors (GIST), glioma, glioblastoma, soft tissue sarcoma, germ cell tumors, childhood central nervous system germ cell tumors, childhood extracranial germ cell tumors, extragonadal germ cell tumors, ovarian germ cell tumors, testicular cancer, gestational trophoblastic disease, hairy cell leukemia, head and neck cancer, heart tumors, hepatocellular cancer, histiocytosis, Langerhans cell, Hodgkin lymphoma, hypopharyngeal cancer, intraocular melanoma, islet cell tumors, pancreatic neuroendocrine tumors, kidney (renal cell) cancer, Langerhans cell histiocytosis, laryngeal cancer, head and neck cancer, leukemia, lip and oral cavity cancer, liver cancer, lung cancer (including non-small cell, small cell, pleuropulmonary blastoma, and/or tracheobronchial tumor), lymphoma, male breast cancer, melanoma, intraocular melanoma, Merkel cell carcinoma, malignant mesothelioma, metastatic cancer, metastatic squamous neck cancer with occult primary, midline tract carcinoma with nut gene changes, mouth cancer, multiple endocrine neoplasia syndromes, multiple myeloma/plasma cell neoplasms, mycosis fungoides, myelodysplastic syndromes, myelodysplastic and/or myeloproliferative neoplasms, nasal cavity and paranasal sinus cancer, nasopharyngeal cancer, neuroblastoma, oral cancer, lip and oral cavity cancer, oropharyngeal, osteosarcoma, undifferentiated pleomorphic sarcoma of bone treatment, ovarian cancer, pancreatic cancer, pancreatic neuroendocrine tumors, islet cell tumors, papillomatosis, paraganglioma, paranasal sinus and nasal cavity cancer, parathyroid cancer, penile cancer, pharyngeal cancer, pheochromocytoma, pituitary tumor, plasma cell neoplasm/multiple myeloma, pleuropulmonary blastoma, pregnancy and breast cancer, primary central nervous system (CNS) lymphoma, primary peritoneal cancer, prostate cancer, rare cancers of childhood, rectal cancer, recurrent cancer, renal cell cancer, retinoblastoma, rhabdomyosarcoma, salivary gland cancer, sarcoma, childhood rhabdomyosarcoma, childhood vascular tumors, osteosarcoma, soft tissue sarcoma, uterine sarcoma, skin cancer, small cell lung cancer, small intestine cancer, soft tissue sarcoma, squamous cell carcinoma of the skin, squamous neck cancer with occult primary, stomach (gastric) cancer, t-cell lymphoma, testicular cancer, throat cancer, nasopharyngeal cancer, oropharyngeal cancer, hypopharyngeal cancer, thymoma and thymic carcinoma, thyroid cancer, tracheobronchial tumors, transitional cell cancer of the renal pelvis and ureter, carcinoma of unknown primary origin, ureter and renal pelvis, transitional cell cancer, urethral cancer, endometrial uterine cancer, uterine sarcoma, vaginal cancer, vascular tumors, vulvar cancer, Wilms tumor and other childhood kidney tumors, or cancer of young adults.

In certain aspects, methods for treating, improving, achieving remission of, and/or reducing the risk of a cancer disclosed herein, involves administering to a subject an immunotherapeutic composition in accordance with the present disclosure. In further aspects, the immunotherapeutic composition may be administered as a pharmaceutical composition according to any appropriate route and regimen. In more aspects, the route or regimen is one that correlates with a positive therapeutic benefit.

In further aspects, the exact amount immunotherapeutic composition administered may vary from patient to patient, depending on one or more factors as is known in the medical arts. Such factors may include, but are not limited to, one or more factors such as species, age, general condition of the patient, the particular composition to be administered, its mode of administration, its mode of activity, the severity of disease, the activity of the specific immunotherapeutic composition employed, the specific pharmaceutical composition administered, the half-life of the composition after administration, body weight, sex, diet of the patient, time of administration, route of administration, rate of excretion of the specific immunotherapeutic composition employed, duration of the treatment, and any other therapeutic agents used in combination or coincidental with the specific immunotherapeutic composition to be administered.

In certain aspects, compositions of the present disclosure are administered to a subject by any appropriate route known and/or employed by those skilled in the art. In some aspects, compositions of the present disclosure are administered by oral (PO), intravenous (IV), intramuscular (IM), intra-arterial, intramedullary, intrathecal, subcutaneous (SQ), intraventricular, transdermal, interdermal, intradermal, rectal (PR), vaginal, intraperitoneal (IP), intragastric (IG), topical (e.g., by powders, ointments, creams, gels, lotions, and/or drops), mucosal, intranasal, buccal, enteral, intravitreal, sublingual, by intratracheal instillation, bronchial instillation, and/or inhalation, as an oral spray, nasal spray, aerosol, and/or through a portal vein catheter.

In certain aspects, an immunotherapeutic composition in accordance with the present disclosure and/or pharmaceutical compositions thereof may be administered intravenously, for example, by intravenous infusion. In further aspects, an immunotherapeutic composition in accordance with the present disclosure and/or pharmaceutical compositions thereof may be administered by intramuscular injection. In more aspects, an immunotherapeutic composition in accordance with the present disclosure and/or pharmaceutical compositions thereof may be administered by intratumoral injection. In certain aspects, an immunotherapeutic composition in accordance with the present disclosure and/or pharmaceutical compositions thereof may be administered by subcutaneous injection. In further aspects, an immunotherapeutic composition in accordance with the present disclosure and/or pharmaceutical compositions thereof may be administered via portal vein catheter. In more aspects, the disclosure encompasses the delivery of an immunotherapeutic composition in accordance with the present disclosure and/or pharmaceutical compositions thereof by any appropriate route taking into consideration likely advances in the art of drug delivery.

In certain aspects, the desired dosage may be delivered only once. In certain aspects, the desired dosage may be delivered once per day, more than once per day, once every other day, once every third day, once every week, once every two weeks, once every three weeks, once every four weeks, once every two months, once every six months, or once every twelve months. In certain aspects, the desired dosage may be delivered using multiple administrations (e.g., two, three, four, five, six, seven, eight, nine, ten, eleven, twelve, thirteen, fourteen, or more administrations). In certain aspects, the desired dosage may be delivered using one or more administrations during an initial period of time, followed by a period of time in which no dosage is administered.

In further aspects, an immunotherapeutic composition in accordance with the present disclosure may be utilized for prophylactic applications. In more aspects, prophylactic applications involve systems and methods for inhibiting progression of, and/or delaying the onset of a cancer in individuals susceptible to and/or displaying symptoms of the cancer.

In certain aspects, an immunotherapeutic composition in accordance with the present disclosure is administered to a target cell in vivo. In other aspects, an immunotherapeutic composition in accordance with the present disclosure is administered to a target cell ex vivo. In additional aspects, an immunotherapeutic composition in accordance with the present disclosure is administered to a target cell ex vivo, then the target cell is re-introduced into an organism. In some such aspects, the target cell is cultured into multiple progeny cells ex vivo before being re-introduced in an organism. In more aspects, the organism is a human. In certain aspects, the target cell was originally derived from the organism to which it is re-introduced. In other aspects, the target cell was originally derived from a different organism to which it is re-introduced.

In certain aspects, an immunotherapeutic composition in accordance with the present disclosure and and/or pharmaceutical compositions thereof are employed in combination therapies for treating or reducing the risk of a cancer. In such aspects, administration can be in combination with one or more additional therapeutic agents. As used herein, the phrases “combination therapy,” “combined with,” “in combination,” and the like, refer to the use of more than one medication or treatment simultaneously to increase the response. In certain aspects, an immunotherapeutic composition in accordance with the present disclosure and and/or pharmaceutical compositions thereof are administered concurrently with, prior to, or subsequent to, one or more other desired therapeutics or medical procedures. In certain aspects, an immunotherapeutic composition in accordance with the present disclosure and/or pharmaceutical compositions thereof are administered in combination together in a single composition or administered separately in different compositions.

In certain aspects, the particular combination of therapies to employ in a combination regimen generally take into account compatibility of the desired therapeutics and/or procedures, and the desired therapeutic effect to be achieved. In further aspects, the therapies employed may achieve a desired effect for the same purpose (e.g., an immunotherapeutic composition in accordance with the present disclosure which is useful for treating, and/or delaying the onset of a cancer may be administered concurrently with another therapeutic agent which is also useful for treating, and/or delaying the onset of the cancer), or they may achieve different effects. In further aspects, the combination of therapies employed may achieve the same or a substantially similar desired effect for the same cancer; may achieve the same or a substantially similar desired effect for one or more different cancers; may achieve different desired effects for the same cancer; or may achieve different desired effects for one or more different cancers.

In additional aspects, the delivery of an immunotherapeutic composition in accordance with the present disclosure as a pharmaceutical composition is in combination with one or more additional components that may improve the bioavailability of an immunotherapeutic composition, reduce and/or modify its metabolism, inhibit its excretion, and/or modify its distribution in the body.

In certain aspects, combination therapy may involve administrations of a plurality of immunotherapeutic compositions in accordance with the present disclosure. In further aspects, combination therapy may involve administrations of a plurality of immunotherapeutic compositions that treat, improve, achieve remission of, and/or reduce the risk of a single type of cancer. In more aspects, combination therapy can be a plurality of immunotherapeutic compositions that treat, improve, achieve remission of, and/or reduce the risk of multiple types of cancers.

In certain aspects, an immunotherapeutic composition in accordance with the present disclosure is combined with at least one pharmaceutically acceptable excipient, in the form of a pharmaceutical composition. As used herein, “pharmaceutical composition” refers to a formulation containing an active ingredient, and optionally a pharmaceutically acceptable carrier, diluent or excipient. The term “active ingredient” can interchangeably refer to an “effective ingredient,” and is meant to refer to any agent that is capable of inducing a sought-after effect upon administration. Examples of active ingredient include, but are not limited to, chemical compound, drug, therapeutic agent, small molecule, and the like. In certain aspects of the present disclosure, the active ingredient is an immunotherapeutic composition as disclosed herein.

By “pharmaceutically acceptable” it is meant the carrier, diluent or excipient must be compatible with the other ingredients of the formulation and not deleterious to the recipient thereof, nor to the activity of the active ingredient of the formulation. Pharmaceutical compositions described herein may be prepared by any method known or hereafter developed in the art of pharmacology. Pharmaceutically acceptable carriers, excipients or stabilizers are known in the art, for example as described J. P. Remington & A. Osol, Remington's Pharmaceutical Sciences, 16th edition (1980) or J. P. Remington & P. Beringer, Remington: The Science and Practice of Pharmacy, 21st Edition (2006), both of which are herein incorporated by reference with respect to the pharmaceutically acceptable carriers, excipients and stabilizers provided therein.

In certain aspects, pharmaceutically acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, and m-cresol); low molecular weight peptides (less than about 10 amino acid residues); proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrin; chelating agents such as E DTA; sugars such as sucrose, mannitol, dextrin, trehalose or sorbitol; salt-forming counter-ions such as sodium; metal complexes (for example, Zn-protein complexes); and/or non-ionic surfactants such as TWEEN™, PLURONICS™ or polyethylene glycol (PEG). Examples of carrier include, but are not limited to, liposome, nanoparticles, ointment, micelles, microsphere, microparticle, cream, emulsion, and gel. Examples of excipients include, but are not limited to, anti-adherents such as magnesium stearate, binders such as saccharides and their derivatives (sucrose, lactose, starches, cellulose, sugar alcohols and the like) protein like gelatin and synthetic polymers, lubricants such as talc and silica, and preservatives such as antioxidants, vitamin A, vitamin E, vitamin C, retinyl palmitate, selenium, cysteine, methionine, citric acid, sodium sulfate and parabens. Examples of diluents include, but are not limited to, water, alcohol, saline solution, glycol, mineral oil and dimethyl sulfoxide (DMSO). In further aspects, the pharmaceutical compositions include one or more additional therapeutically or biologically active substances.

In certain aspects, the pharmaceutical compositions are useful in medicine or the manufacture of medicaments. In further aspects, the pharmaceutical compositions are useful in one or more of the therapeutic applications disclosed herein, for example, in an individual suffering from an autoimmune disorder. In additional aspects, the pharmaceutical compositions are formulated for administration to a human subject.

In certain aspects, the pharmaceutical composition is in a sterile injectable form (e.g., a form that is suitable for subcutaneous injection or intravenous infusion). In more aspects, the pharmaceutical composition is in a liquid dosage form that is suitable for injection. In further aspects, the pharmaceutical composition is in a powder (e.g., lyophilized and/or sterilized), optionally under vacuum, which is reconstituted with an aqueous diluent (e.g., water; buffer; salt solution, and the like) prior to injection. In additional aspects, the pharmaceutical composition is diluted and/or reconstituted in an aqueous diluent (e.g., water, sodium chloride solution, sodium acetate solution, benzyl alcohol solution, phosphate buffered saline, and the like). In certain aspects, the pharmaceutical composition is in a form that can be refrigerated and/or frozen. In further aspects, the pharmaceutical composition is in a form that cannot be refrigerated and/or frozen. In certain aspects, the pharmaceutical composition is a reconstituted solution and/or liquid dosage form which can be stored for a certain period of time after reconstitution (e.g., 2 hours, 12 hours, 24 hours, 2 days, 5 days, 7 days, 10 days, 2 weeks, a month, two months, or longer).

In certain aspects, preparatory methods for pharmaceutical compositions include bringing the active ingredient (e.g., an immunotherapeutic composition in accordance with the present disclosure) into association with one or more pharmaceutically acceptable excipients and then shaping and/or packaging the product into a desired single- or multi-dose unit. A pharmaceutical composition in accordance with the disclosure may be prepared, packaged in bulk, packaged as a single unit dose, and/or packaged as a plurality of single unit doses. As used herein, a “unit dose” refers to a discrete amount of the pharmaceutical composition including a predetermined amount of the active ingredient. The amount of the active ingredient is generally equal to a dose that would be administered to a subject and/or a convenient fraction of such a dose such as, for example, one-half or one-third of such a dose. The relative amounts of active ingredient, pharmaceutically acceptable excipient, and/or any additional ingredients in a pharmaceutical composition in accordance with the disclosure may vary, depending upon the identity, size, and/or condition of the subject treated and/or depending upon the route by which the composition is to be administered. In certain aspects, for example, the composition may include between about 0.1% to 100% (w/w) of an active ingredient.

In another aspect, the present disclosure includes kits that are useful for carrying out the methods of the present disclosure. The components contained in the kit depend on a number of factors, including the particular application (e.g., the particular route of administration to be employed, or the particular cancer to be treated). In certain aspects, the present disclosure provides a kit for administering an immunotherapeutic composition in accordance with the present disclosure to treat a type of cancer disclosed herein. In some such aspects, the kit further includes instructions for administration. In certain aspects, the kits contain one or more immunotherapeutic compositions. In certain aspects, the kit includes a number of unit doses of a pharmaceutical composition containing an immunotherapeutic composition. In additional aspects, kits for use in accordance with the present disclosure include instructions (e.g., for administration, for storage, and the like), buffers and/or other reagents. In some such aspects, the kit includes (i) at least one immunotherapeutic composition, (ii) a syringe, needle, applicator, or the like for administration of the at least one immunotherapeutic composition to a subject, and (iii) instructions for use. In further aspects, the kit includes a treatment schedule designating when the unit dosages are to be administered. In more aspects, placebo dosages, either in a form similar to or distinct from the dosages of the pharmaceutical compositions, are included. In certain aspects, kits include one or more containers so that certain of the individual components or reagents may be separately housed. In certain aspects, kits may include a means for enclosing the individual containers in relatively close confinement for commercial sale, e.g., a plastic box, in which instructions, packaging materials such as styrofoam, and the like, may be enclosed.
Chen D S, Mellman I. Oncology meets immunology: the cancer-immunity cycle. Immunity. 2013 Jul. 25; 39(1):1-10. doi: 10.1016/j.immuni.2013.07.012. PMID: 23890059.
Zheng M, Williams E P, Malireddi R K S, Karki R, Banoth B, Burton A, Webby R, Channappanavar R, Jonsson C B, Kanneganti T D. Impaired NLRP3 inflammasome activation/pyroptosis leads to robust inflammatory cell death via caspase-8/RIPK3 during coronavirus infection. J Biol Chem. 2020 Oct. 9; 295(41):14040-14052. doi: 10.1074/jbc.R A 120.015036. E pub 2020 Aug. 6. PMID: 32763970; PMCID: PMC7549031.
Lee S, Channappanavar R, Kanneganti T D. Coronaviruses: Innate Immunity, Inflammasome Activation, Inflammatory Cell Death, and Cytokines. Trends Immunol. 2020 December; 41(12):1083-1099. doi: 10.1016/j.it.2020.10.005. Epub 2020 Oct. 15. PMID: 33153908; PMCID: PMC7561287.
Agudo J, Ruzo A, Park E S, Sweeney R, Kana V, Wu M, Zhao Y, Egli D, Merad M, Brown B D. GFP-specific CD8 T cells enable targeted cell depletion and visualization of T-cell interactions. Nat Biotechnol. 2015 December; 33(12):1287-1292. doi: 10.1038/nbt.3386. Epub 2015 Nov. 2. PMID: 26524661; PMCID: PMC4675673.
Hoffman R M. Application of GFP imaging in cancer. Lab Invest. 2015 April; 95(4):432-52. doi: 10.1038/labinvest.2014.154. Epub 2015 Feb. 16. PMID: 25686095; PMCID: PMC4383682.
Liu B L, Robinson M, Han Z Q, Branston R H, English C, Reay P, McGrath Y, Thomas S K, Thornton M, Bullock P, Love C A, Coffin R S. ICP34.5 deleted herpes simplex virus with enhanced oncolytic, immune stimulating, and anti-tumour properties. Gene Ther. 2003 February; 10(4):292-303. doi: 10.1038/sj.get.3301885. PMID: 12595888.
Rayamajhi M, Zhang Y, Miao E A. Detection of pyroptosis by measuring released lactate dehydrogenase activity. Methods Mol Biol. 2013; 1040:85-90. doi: 10.1007/978-1-62703-523-1_7. PMID: 23852598; PMCID: PMC3756820.
HuanzhouXu, Siddhi A. Chitre, Ibukun A. Akinyemi, Julia C. Loeb, John A. Lednicky, M ichael T. McIntosh, Sumita Bhaduri-McIntosh. SARS-CoV-2 viroporin triggers the NLRP3 inflammatory pathway. bioRxiv 2020.10.27.357731; doi: https://doi.org/10.1101/2020.10.27.357731.
Shah A. Novel Coronavirus-Induced NLRP3 Inflammasome Activation: A Potential Drug Target in the Treatment of COVID-19. Front Immunol. 2020 May 19; 11:1021. doi: 10.3389/fimmu.2020.01021. PMID: 32574259; PMCID: PMC7248552.
Gu H, Chen Q, Yang G, He L, Fan H, Deng Y Q, Wang Y, Teng Y, Zhao Z, Cui Y, Li Y, Li X F, Li J, Zhang N N, Yang X, Chen S, Guo Y, Zhao G, Wang X, Luo D Y, Wang H, Yang X, Li Y, Han G, He Y, Zhou X, Geng S, Sheng X, Jiang S, Sun S, Qin C F, Zhou Y. Adaptation of SARS-CoV-2 in BALB/c mice for testing vaccine efficacy. Science. 2020 Sep. 25; 369(6511):1603-1607. doi: 10.1126/science.abc4730. Epub 2020 Jul. 30. PMID: 32732280; PMCID: PMC7574913.
Bewley K R, Coombes N S, Gagnon L, McInroy L, Baker N, Shaik I, St-Jean J R, St-Amant N, Buttigieg K R, Humphries H E, Godwin K J, Brunt E, Allen L, Leung S, Brown P J, Penn E J, Thomas K, Kulnis G, Hallis B, Carroll M, Funnell S, Charlton S. Quantification of SARS-CoV-2 neutralizing antibody by wild-type plaque reduction neutralization, microneutralization and pseudotyped virus neutralization assays. Nat Protoc. 2021 June; 16(6):3114-3140. doi: 10.1038/s41596-021-00536-y. Epub 2021 Apr. 23. PMID: 33893470.
Nie J, Li Q, Wu J, Zhao C, Hao H, Liu H, Zhang L, Nie L, Qin H, Wang M, Lu Q, Li X, Sun Q, Liu J, Fan C, Huang W, Xu M, Wang Y. Quantification of SARS-CoV-2 neutralizing antibody by a pseudotyped virus-based assay. Nat Protoc. 2020 November; 15(11):3699-3715. doi: 10.1038/s41596-020-0394-5. Epub 2020 Sep. 25. PMID: 32978602.

It is to be understood that this disclosure is not limited to particular compositions, methods, and experimental conditions described, as such compositions, methods, and conditions may vary. It is also to be understood that the terminology used herein is for purposes of describing particular embodiments only, and is not intended to be limiting, since the scope of the present disclosure will be limited only in the appended claims.

All publications, patents, and patent applications mentioned in this specification are herein incorporated by reference to the same extent as if each individual publication, patent, or patent application was specifically and individually indicated to be incorporated by reference.

1. A pharmaceutical composition comprising an autologous cancer cell isolated from a subject, wherein the autologous cancer cell comprises:
a) a recombinant protein or peptide or nucleic acid encoding a recombinant protein or peptide that induces an immune response;
b) a recombinant protein or peptide or nucleic acid encoding a recombinant protein or peptide that induces an inflammatory cell death response;
c) a recombinant protein or peptide or nucleic acid encoding a recombinant protein or peptide that induces the activation and persistence of antigen-presenting cells and cytotoxic T cells within the microenvironment of a tumor; or
d) a recombinant protein or peptide or nucleic acid encoding a recombinant protein or peptide that induces the formation of tumor-associated tertiary lymphoid structures.

2. The pharmaceutical composition of claim 1, wherein the recombinant protein or peptide that induces an immune response is selected from the group consisting of:
a) SARS-Cov-2 Spike (S);
b) SARS-Cov-2 Envelope (E);
c) SARS-Cov-2 Membrane (M);
d) SARS-Cov-2 Nucleocapsid (N);
e) SARS-CoV-2 Spike S-2P and RBD antigen;
f) SARS-Cov-2 ORF3a;
g) SARS-Cov-2 ORF7a;
h) SARS-Cov-2 ORF8;
i) SARS-Cov-2 Replicase 1AB;
j) Enhanced Green Fluorescent Protein (EGFP); and
k) Influenza Hemagglutinin (HA).

3. The pharmaceutical composition of claim 1, wherein the recombinant protein or peptide that induces an inflammatory cell death response is selected from the group consisting of:
a) Gasdermin D (GSDMD);
b) Gasdermin E (GSDME);
c) Absent In Melanoma-2 (AIM2);
d) Interleukin 33 (IL33);
e) Thioredoxin-Interacting Protein (TXNIP);
f) Interleukin 1 Receptor Associated Kinase 1 (IRAK1); and
g) NLR Family Pyrin Domain Containing 3 (NLRP3).

4. The pharmaceutical composition of claim 1, wherein the recombinant protein or peptide that induces the activation and persistence of antigen-presenting cells and cytotoxic T cells within the microenvironment of a tumor and the recombinant protein or peptide that induces the formation of tumor-associated tertiary lymphoid structures is selected from the group consisting of:
a) Chemokine Ligand 9 (CXCL9);
b) Chemokine Ligand 10 (CXCL10);
c) Chemokine Ligand 12 (CXCL12);
d) Chemokine Ligand 13 (CXCL13);
e) C-C Motif Chemokine Ligand 19 (CCL19);
f) C-C Motif Chemokine Ligand 21 (CCL21);
g) Interleukin 1 Beta (IL1β);
h) Interleukin 4 (IL4);
i) Interleukin 4 Receptor (IL4R);
j) Interleukin 21 (IL21);
k) Interleukin 22 (IL22);
l) Interleukin 22 Receptor (IL22R);
m) Interleukin 23;
n) Interleukin 13 (IL13);
o) Lymphotoxin Beta Receptor (LTOR);
p) Beta-2-Microglobulin (B2M);
q) Programmed Death-Ligand 1 (PD-L1);
r) Integrin Associated Protein (CD47);
s) Transforming Growth Factor Beta Receptor 1 (TGFβRI)-ectodomain, soluble;
t) Transforming Growth Factor Beta Receptor II (TGFβRII)-ectodomain, soluble;
u) Transforming Growth Factor Beta Receptor III (TGFβRIII)-ectodomain, soluble;
v) a protein consisting of an ectodomain of at least one protein selected from the group consisting of Transforming Growth Factor Beta Receptor I (TGFβRI), Transforming Growth Factor Beta Receptor II (TGFβRII), and Transforming Growth Factor Beta Receptor III (TGFβRIII);
w) Mothers Against Decapentaplegic Homolog 2 (SMAD2);
x) Mothers Against Decapentaplegic Homolog 3 (SMAD3)
y) Tumor Necrosis Factor Alpha (TNFα); and
z) Interferon Gamma (IFNγ).

5. The pharmaceutical composition of claim 1, wherein the autologous cancer cell is contacted with a mixture of cytokines and chemokines.

6. The pharmaceutical composition of claim 4, wherein at least one of the recombinant proteins or peptides is Tumor Necrosis Factor Alpha (TNFα) or Interferon Gamma (IFNγ).

7. A pharmaceutical composition formulated for delivery with a lipid nanoparticles, electroporation, or other delivery mechanisms, comprising the autologous cancer cell of claim 1.

8. A method for treating cancer in a subject, comprising administering to the subject the pharmaceutical composition of claim 1.

9. A method for treating a benign tumor in a subject, comprising administering to the subject the pharmaceutical composition of claim 1.

10. A pharmaceutical composition comprising an allogenic cell comprising:
a) a recombinant protein or peptide or nucleic acid encoding a recombinant protein or peptide that induces an immune response;
b) a recombinant protein or peptide or nucleic acid encoding a recombinant protein or peptide that induces an inflammatory cell death response;
c) a recombinant protein or peptide or nucleic acid encoding a recombinant protein or peptide that induces the activation and persistence of antigen-presenting cells and cytotoxic T cells within the microenvironment of a tumor; or
d) a recombinant protein or peptide or nucleic acid encoding a recombinant protein or peptide that induces the formation of tumor-associated tertiary lymphoid structures.

11. The pharmaceutical composition of claim 10, wherein the recombinant protein or peptide that induces an immune response is selected from the group consisting of:
a) SARS-Cov-2 Spike (S);
b) SARS-Cov-2 Envelope (E);
c) SARS-Cov-2 Membrane (M);
d) SARS-Cov-2 Nucleocapsid (N);
e) SARS-CoV-2 Spike S-2P and RBD antigen;
f) SARS-Cov-2 ORF3a;
g) SARS-Cov-2 ORF7a;
h) SARS-Cov-2 ORF8;
i) SARS-Cov-2 Replicase 1AB;
j) Enhanced Green Fluorescent Protein (EGFP); and
k) Influenza Hemagglutinin (HA).

12. The pharmaceutical composition of claim 10, wherein the recombinant protein or peptide that induces an inflammatory cell death response is selected from the group consisting of:
a) Gasdermin D (GSDMD);
b) Gasdermin E (GSDME);
c) Absent In Melanoma-2 (AIM2);
d) Interleukin 33 (IL33);
e) Thioredoxin-Interacting Protein (TXNIP);
f) Interleukin 1 Receptor Associated Kinase 1 (IRAK1); and
g) NLR Family Pyrin Domain Containing 3 (NLRP3).

13. The pharmaceutical composition of claim 10, wherein the recombinant protein or peptide that induces the activation and persistence of antigen-presenting cells and cytotoxic T cells within the microenvironment of a tumor and the recombinant protein or peptide or nucleic acid encoding the recombinant protein or peptide that induces that induce the formation of tumor-associated tertiary lymphoid structures is selected from the group consisting of:
a) Chemokine Ligand 9 (CXCL9);
b) Chemokine Ligand 10 (CXCL10);
c) Chemokine Ligand 12 (CXCL12);
d) Chemokine Ligand 13 (CXCL13);
e) C-C Motif Chemokine Ligand 19 (CCL19);
f) C-C Motif Chemokine Ligand 21 (CCL21);
g) Interleukin 1 Beta (IL10);
h) Interleukin 4 (IL4);
i) Interleukin 4 Receptor (IL4R);
j) Interleukin 21 (IL21);
k) Interleukin 22 (IL22);
l) Interleukin 22 Receptor (IL22R);
m) Interleukin 23;
n) Interleukin 13 (IL13);
o) Lymphotoxin Beta Receptor (LTOR);
p) Beta-2-Microglobulin (B2M);
q) Programmed Death-Ligand 1 (PD-L1);
r) Integrin Associated Protein (CD47);
s) Transforming Growth Factor Beta Receptor I (TGFβRI)-ectodomain, soluble;
t) Transforming Growth Factor Beta Receptor II (TGFβRII)-ectodomain, soluble;
u) Transforming Growth Factor Beta Receptor III (TGFβRIII)-ectodomain, soluble;
v) a fusion protein consisting of an ectodomain of at least one protein selected from the group consisting of: Transforming Growth Factor Beta Receptor I (TGFβRI), Transforming Growth Factor Beta Receptor II (TGFβRII), and Transforming Growth Factor Beta Receptor III (TGFβRIII);
w) Mothers Against Decapentaplegic Homolog 2 (SMAD2); and
x) Mothers Against Decapentaplegic Homolog 3 (SMAD3).

14. The pharmaceutical composition of claim 10, wherein the allogenic cell is contacted with an autologous cancer cell from a subject, wherein the autologous cancer cell is first contacted with a mixture of cytokines and chemokines.

15. A pharmaceutical composition formulated for delivery using lipid nanoparticles, electroporation, or other suitable delivery mechanisms, comprising the allogenic cell of claim 10.

16. A method for treating cancer in a subject, comprising administering to the subject the pharmaceutical composition of claim 10.

17. A method for treating a benign tumor in a subject, comprising administering to the subject the pharmaceutical composition of claim 10.

18. A pharmaceutical composition comprising a recombinant virus selected from the group consisting of:
a) a recombinant protein or peptide or nucleic acid encoding a recombinant protein or peptide that induces an immune response;
b) a recombinant protein or peptide or nucleic acid encoding a recombinant protein or peptide that induces an inflammatory cell death response;
c) a recombinant protein or peptide or nucleic acid encoding a recombinant protein or peptide that induces the activation and persistence of antigen-presenting cells and cytotoxic T cells within the microenvironment of a tumor; and
d) a recombinant protein or peptide or nucleic acid encoding a recombinant protein or peptide that induces the formation of tumor-associated tertiary lymphoid structures.

19. The pharmaceutical composition of claim 18, wherein the nucleic acid encoding the recombinant protein or peptide that induces an immune response encodes a protein or peptide selected from the group consisting of:
a) SARS-Cov-2 Spike (S);
b) SARS-Cov-2 Envelope (E);
c) SARS-Cov-2 Membrane (M);
d) SARS-Cov-2 Nucleocapsid (N);
e) SARS-CoV-2 Spike S-2P and RBD antigen
f) SARS-Cov-2 ORF3a;
g) SARS-Cov-2 ORF7a;
h) SARS-Cov-2 ORF8;
i) SARS-Cov-2 Replicase 1AB;
j) Enhanced Green Fluorescent Protein (EGFP); and
k) Influenza Hemagglutinin (HA).

20. The pharmaceutical composition of claim 18, wherein the nucleic acid encoding the recombinant protein or peptide that induces an inflammatory cell death response encodes a protein or peptide selected from the group consisting of:
a) Gasdermin D (GSDMD);
b) Gasdermin E (GSDME);
c) Absent In Melanoma-2 (AIM2);
d) Interleukin 33 (IL33);
e) Thioredoxin-Interacting Protein (TXNIP);
f) Interleukin 1 Receptor Associated Kinase 1 (IRAK1); and
g) NLR Family Pyrin Domain Containing 3 (NLRP3).

21. The pharmaceutical composition of claim 18, wherein the recombinant protein or peptide that induces the activation and persistence of antigen-presenting cells and cytotoxic T cells within the microenvironment of a tumor and the recombinant protein or peptide that induces the formation of tumor-associated tertiary lymphoid structures is selected from the group consisting of:
a) Chemokine Ligand 9 (CXCL9);
b) Chemokine Ligand 10 (CXCL10);
c) Chemokine Ligand 12 (CXCL12);
d) Chemokine Ligand 13 (CXCL13);
e) C-C Motif Chemokine Ligand 19 (CCL19);
f) C-C Motif Chemokine Ligand 21 (CCL21);
g) Interleukin 1 Beta (IL1β);
h) Interleukin 4 (IL4);
i) Interleukin 4 Receptor (IL4R);
j) Interleukin 21 (IL21);
k) Interleukin 22 (IL22);
l) Interleukin 22 Receptor (IL22R);
m) Interleukin 23;
n) Interleukin 13 (IL13);
o) Lymphotoxin Beta Receptor (LTOR);
p) Beta-2-Microglobulin (B2M);
q) Programmed Death-Ligand 1 (PD-L1);
r) Integrin Associated Protein (CD47);
s) Transforming Growth Factor Beta Receptor I (TGFβRI)-ectodomain, soluble;
t) Transforming Growth Factor Beta Receptor II (TGFβRII)-ectodomain, soluble;
u) Transforming Growth Factor Beta Receptor III (TGFβRIII)-ectodomain, soluble;
v) A fusion protein consisting of an ectodomain of at least one protein selected from the group consisting of: Transforming Growth Factor Beta Receptor I (TGFβRI), Transforming Growth Factor Beta Receptor II (TGFβRII), and Transforming Growth Factor Beta Receptor III (TGFβRIII);
w) Mothers Against Decapentaplegic Homolog 2 (SMAD2); and
x) Mothers Against Decapentaplegic Homolog 3 (SMAD3).

22. A pharmaceutical composition for delivery to a subject using lipid nanoparticles, electroporation, or other suitable delivery mechanisms, comprising the recombinant virus of claim 18.

23. A method for treating cancer in a subject, comprising administering to the subject the pharmaceutical composition of claim 18.

24. A method for treating a benign tumor in a subject, comprising administering to the subject the pharmaceutical composition of claim 18.

25. A pharmaceutical composition comprising an allogenic T cell, wherein the allogenic T cell comprises:
a) a chimeric antigen receptor (CAR) or an antigen specific T cell receptor (TCR) that makes the T cells antigen specific;
b) a recombinant protein or peptide or mRNA encoding a recombinant protein or peptide that induces the activation and persistence of antigen-presenting cells and cytotoxic T cells within the microenvironment of a tumor; and
c) a recombinant protein or peptide or mRNA encoding a recombinant protein or peptide that induces that induce the formation of tumor-associated tertiary lymphoid structures.

26. The pharmaceutical composition of claim 25, wherein the chimeric antigen receptor (CAR) or an antigen specific T cell receptor (TCR) is specific for a protein selected from the group consisting of:
a) SARS-Cov-2 Spike (S);
b) SARS-Cov-2 Envelope (E);
c) SARS-Cov-2 Membrane (M);
d) SARS-Cov-2 Nucleocapsid (N);
e) SARS-CoV-2 Spike S-2P and RBD antigen;
f) SARS-Cov-2 ORF3a;
g) SARS-Cov-2 ORF7a;
h) SARS-Cov-2 ORF8;
i) SARS-Cov-2 Replicase 1AB;
j) Enhanced Green Fluorescent Protein (EGFP); and
k) Influenza Hemagglutinin (HA).

27. The pharmaceutical composition of claim 25, wherein the recombinant protein or peptide that induces the activation and persistence of antigen-presenting cells and cytotoxic T cells within the microenvironment of a tumor and the recombinant protein or peptide that induces the formation of tumor-associated tertiary lymphoid structures is selected from the group consisting of:
a) Chemokine Ligand 9 (CXCL9);
b) Chemokine Ligand 10 (CXCL10);
c) Chemokine Ligand 12 (CXCL12);
d) Chemokine Ligand 13 (CXCL13);
e) C-C Motif Chemokine Ligand 19 (CCL19);
f) C-C Motif Chemokine Ligand 21 (CCL21);
g) Interleukin 1 Beta (IL1β);
h) Interleukin 4 (IL4);
i) Interleukin 4 Receptor (IL4R);
j) Interleukin 21 (IL21);
k) Interleukin 22 (IL22);
l) Interleukin 22 Receptor (IL22R);
m) Interleukin 23;
n) Interleukin 13 (IL13);
o) Lymphotoxin Beta Receptor (LTOR);
p) Beta-2-Microglobulin (B2M);
q) Programmed Death-Ligand 1 (PD-L1);
r) Integrin Associated Protein (CD47);
s) Transforming Growth Factor Beta Receptor I (TGFβRI)-ectodomain, soluble;
t) Transforming Growth Factor Beta Receptor II (TGFβRII)-ectodomain, soluble;
u) Transforming Growth Factor Beta Receptor III (TGFβRIII)-ectodomain, soluble;
v) A fusion protein comprising an ectodomain of at least one protein selected from the group consisting of: Transforming Growth Factor Beta Receptor I (TGFβRI), Transforming Growth Factor Beta Receptor II (TGFβRII), and Transforming Growth Factor Beta Receptor III (TGFβRIII);
w) Mothers Against Decapentaplegic Homolog 2 (SMAD2); and
x) Mothers Against Decapentaplegic Homolog 3 (SMAD3).

28. A pharmaceutical composition suitable for delivery to a subject using lipid nanoparticles, electroporation, or other suitable delivery mechanisms, comprising the allogenic cell of claim 25.

29. A method for treating cancer in a subject, comprising administering to the subject the pharmaceutical composition of claim 25.

30. A method for treating a benign tumor in a subject, comprising administering to the subject the pharmaceutical composition of claim 25.

31-35. (canceled)
